SEQUENCE LISTING (5 sequences)

SEQ ID NO: 1 - DNA, 642 bp, Homo sapiens
  CDS (47)..(535)
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggagccctattaaattatataaagaaata

SEQ ID NO: 2 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - protein, 10 aa, Artificial Sequence
  Description of Artificial Sequence  Synthetic Peptide
CGDPKHPKSF

SEQ ID NO: 4 - DNA, 24 bp, Artificial Sequence
  Description of Artificial Sequence  Synthetic Primer
tcgtcgttttgtcgttttgtcgtt

SEQ ID NO: 5 - DNA, 20 bp, Artificial Sequence
  Description of Artificial Sequence  Synthetic Primer
gggggagcatgctggggggg

(19) United States
(12) Patent Application Publication
(10) Pub. No.: US 2007/0071717 A1
(43) Pub. Date: Mar. 29, 2007

(54) Treatment of B cells with IL-21 and B cell activators induces Granzyme B production

(75) Inventors: George Weiner, Iowa City, IA (US); Bernd Jahrsdorfer, Neusaess (DE)

Correspondence Address: FULBRIGHT & JAWORSKI L.L.P., 600 CONGRESS AVE., SUITE 2400, AUSTIN, TX, 78701, US

(73) Assignee: University of Iowa Research Foundation

(21) Appl. No.: 11/517,192

(22) Filed: Sep. 7, 2006

Related U.S. Application Data
(60) Provisional application No. 60/786,642, filed on Mar. 28, 2006.
(60) Provisional application No. 60/714,755, filed on Sep. 7, 2005.

(51) Int. Cl. A61K 48/00; A61K 39/395; A61K 38/20; A61K 38/19

(52) U.S. Cl. 424/85.1; 424/85.2; 424/131.1; 514/44

(57) ABSTRACT

The present invention involves the combined use of IL-21 and TLR agonists such as CpG oligonucleotides in the treatment of B cell cancers and B cell-related immune pathologies such as autoimmune diseases. In addition, it is demonstrated that human B cells produce and secrete varying amounts of Granzyme B in response to IL-21 depending on their activation state, and at the same order of magnitude as those secreted by cytotoxic T lymphocytes. In CpG ODN-treated B-CLL cells, Granzyme B secretion in response to IL-21 can be cytotoxic.

This application claims priority to U.S. Provisional Patent applications having Ser. No. 60/786,642 filed Mar. 28, 2006, entitled “Treatment of B-cells with B-cell activators induces Granzyme B production,” and Ser. No. 60/714,755 filed Sep. 7, 2005, entitled “Treatment of B-cell diseases with IL-21 and TLR agonists,” both of which are incorporated herein by reference in their entirety.

The United States Government may own rights in the present invention pursuant to grant CA097274 and CA077764 from the NIH.

### BACKGROUND OF THE INVENTION

### I. FIELD OF THE INVENTION

The present invention relates generally to the fields of immunology, oncology, cellular biology, and molecular biology. The invention provides for the generation of Granzyme B-secreting cytotoxic B cells and/or the treatment of B cell related diseases by using combinations of IL-21 and a secondary B cell-stimulatory agent, such as toll-like receptor (TLR) agonists.

### II. BACKGROUND

B-lymphocytes or B cells are responsible for humoral immunity. They arise from a separate population of stem cells of the bone marrow than the stem cells that give rise to T cells. These cells undergo multiplication and processing in lymphoid tissue elsewhere than in the thymus gland. In birds, the lymphoid tissue concerned has been located in the gut and called the bursa of Fabricius. In humans, the site is unknown, although there is some evidence to suggest that such processing occurs in the bone marrow itself or in the fetal liver. Lymphocytes processed in this way are called B-lymphocytes after the bursa.

B cells, like T cells, have surface receptors which enable them to recognize the appropriate antigen, but are not so far known to interact to neutralize or destroy the antigen themselves. If the B cell comes into contact with the specific type of antigen to which it is targeted, it divides rapidly to form a clone of identical cells (short-lived plasma cells). The plasma cells produce antibodies and release them into the circulation at the lymph nodes. Some of the activated B cells do not become plasma cells, but instead they turn into memory cells which continue to produce small amounts of the antibody long after the infection has been overcome. Antibody circulates as part of the gamma globulin fraction of the blood plasma. Should the same antigen enter the body again this circulating antibody acts quickly to destroy it, and at the same time memory cells quickly divide to produce new clones of the appropriate type of plasma cell.

Clearly, then B cells and antibodies form a critical part of the human immune response. Unfortunately, Ig production is not always beneficial. It has become more and more evident during recent years that members of the CD5- (formerly Leu-1-) positive, so called B1 cell subset, are involved in the initiation and perpetuation of various autoimmune processes by contributing to the production of self-reactive antibodies. Numerous disease states characterized by excessive or inappropriate immunoglobulin production have been identified, including systemic lupus erythematosus, rheumatoid arthritis, systemic sclerosis, polymyositis, Sjögren's Syndrome, graft rejection, Grave's disease, myasthenia gravis, cancer characterized by hyperimmunoglobulinemia, mononucleosis, and hyper-Ig syndromes. In many cases, the Ig produces attacks on host cell antigens, causing inflammation and tissue destruction. Thus, it would be highly beneficial to identify mechanisms of down-regulating pathologic Ig production, and employing such methods as therapies for the aforementioned disease states.

Like Ig, cytokines are an essential part of the immune response. These peptides are used by immune and inflammatory cells to communicate with each other and to control the milieu interieur in which they operate. Evidence indicates their immense importance in controlling the local and systemic events of the immune response, inflammation, hemopoiesis, healing, and the systemic response to injury. But also as with Ig, their uncontrolled production can lead to devastating disease. For example, IL-1 is associated with joint inflammation, IL-6 is linked to some cancers, and TNFα is a factor in sepsis.

Yet another aberrant B cell disease state involves cancer. There are a number of B cell-related cancers that involve the activation and proliferation of B cells. Also here, the particular subset of B1-cells, finds a particular role in chronic lymphocytic leukemia and mantle cell lymphoma. Though chemotherapy and radiotherapy, along with other biological treatments (steroids, antibodies) are used to treat these cancers, just as with other B cell diseases, improved methods of treatment are needed.

### SUMMARY OF THE INVENTION

Thus in accordance with the present invention, there is provided a method of generating a cytotoxic Granzyme B-producing B cell comprising contacting a B cell with a cytokine, such as IL-21 or IL-10 and one or more of a second agent selected from the group consisting of a TLR agonist, a cytokine, an antigen, anti-idiotype antibody, or an agent that cross-links surface immunoglobulin. A B cell may be a malignant B cell. The TLR agonist may be a CpG ODN, immunostimulatory DNA, immunostimulatory RNA, immunostimulatory oligonucleotides, Imiquimod, Resiquimod, Loxribine, Flagellin, FSL-1 or LPS; the cytokine may be a IL-1, IL-2, IL-3, IL-4, IL-6, IL-7, IL-9, IL-10, IL-12, IL-15, IL-18, IFN-α, IFN-β, IFN-γ, G-CSF, or GM-CSF. The antigen may be a self antigen, a non-self antigen, a peptide antigen, a nucleic acid antigen, a carbohydrate antigen, a cancer antigen, and/or a pathogen antigen. The agent that cross-links surface immunoglobulin may be an anti-Ig antibody, anti-idiotype antibody, or anti-isotype antibody.

Contacting may comprise administration of IL-21 and the second agent to a subject, such as by systemic or intranodal routes. The contacting may occur in vitro, and may further comprise administering the cytotoxic B cells to a subject. The subject may suffer from any cancer including but not limited to B cell malignancies. The subject may suffer from an infectious disease, such as a bacterial, parasitic, fungal or viral infection.

The subject may suffer from an autoimmune disease, such as systemic lupus erythematosus; rheumatoid arthritis; Sjögren's syndrome; systemic sclerosis; polymyositis; grave's disease; myasthenia gravis; autoimmune diabetes (juvenile diabetes or diabetes type I diabetes); mononucleosis; Hyper-IgM, -IgD, or -IgE syndrome; or a hyperimmune disease, such as an anaphylactic reaction, a disease of excess or aberrant cytokine production, an auto-destructive immune response following infection with virus, bacteria, fungi or parasites or an auto-destructive immune response following antibiotic, antiviral, anti-fungal, or anti-parasitic therapy. The method may further comprise treatment of the subject with a standard autoimmune disease therapy or hyperimmune disease therapy.

In still another embodiment, there is provided a method of generating an immune response in a subject comprising providing to the subject a cytotoxic Granzyme B-producing B cell. The immune response may be an anti-tumor immune response, anti-viral immune response, an anti-bacterial immune response, an anti-parasitic immune response, an anti-fungal immune response, or an immune-regulatory response. Providing may comprise administering to the subject one or more cytokine, such as IL-21 or IL-10, and one or more second agent selected from the group consisting of a TLR agonist, a cytokine, an antigen or anti-B cell receptor antibody, or administering to the subject a cytotoxic Granzyme B-producing B cell.

In still yet another embodiment, there is provided a method of inhibiting a T-regulatory response comprising providing to the subject a cytotoxic Granzyme B-producing B cell. Providing may comprise administering to the subject IL-21 and one or more second agent selected from the group consisting of a TLR agonist, a cytokine, an antigen or anti-B cell receptor antibody, or administering to the subject a cytotoxic Granzyme B-producing B cell.

In certain aspects, IL-21 is not the only cytokine that can induce the cytotoxic differentiation pathway in B cells. The inventors have identified at least two cytokine combinations of IL-10+IL-4 and IL-10+IFN-α with similar effects. Another aspect of the invention is that bone marrow stroma cells can also induce B cells to produce granzyme B. This is indicative of the physiological function of the inventors findings since it could represent a way how autoreactive B cells may be deleted in the bone marrow. In another aspect of the invention B cells are able to inhibit the expansion of CD4-positive T cells in the presence of IL-2 1, CpG ODN and B cell receptor.

Other embodiments or the invention include methods of inhibiting a B cell disease comprising contacting an activated or hyperproliferative B cell with IL-21 and a toll-like receptor (TLR) agonist. The TLR agonist may be an immunostimulatory oligodeoxynucleotide (ODN), imiquimod, FSL-1, or loxoribin. The immunostimulatory ODN may be a CpG ODN. The TLR agonist may target TLR1, TLR2, TLR6, TLR7, TLR9, or TLR10. Inhibiting may comprise inhibiting antibody production, inhibiting cytokine production, inhibiting B cell growth, inhibiting B cell division or inducing apoptosis. The cell may be located in an animal, such as a human. The B cell may be subjected to a second therapy, such as chemotherapy, radiotherapy, immune therapy, gene therapy, toxin therapy or surgery, or an anti-inflammatory or immunosuppressive antibody therapy. The B cell may be a B1 B cell.

The B cell disease may be a cancer or an autoimmune disease. The hyperproliferative B cell, optionally a B1 B cell, may be a cancer cell, such as a chronic lymphocytic leukemia cell or mantle cell lymphoma cell. Alternatively, the activated B cell, optionally a B1 B cell, may produce an autoimmune antibody. The IL-21 may be contacted with the B cell at the same time as the TLR agonist, prior to the TLR agonist, or after the TLR agonist. The cell may be contacted with either IL-21 or the TLR agonist a second time or both IL-21 and the TLR agonist a second time. The TLR agonist and IL-21 may be delivered intravenously or subcutaneously. The TLR agonist and IL-21 may be delivered intratumorally, into tumor vasculature or into a post-operative tumor bed. The IL-21 may be provided to the B cell by recombinant expression in a non-B cell or the B cell, for example, by a viral or non-viral expression construct encoding IL-21.

Also provided is a pharmaceutical composition comprising IL-21 and a toll-like receptor (TLR) agonist dispersed in a pharmaceutically acceptable buffer, diluent, or excipient. The TLR agonist may be an immunostimulatory oligonucleotide (ODN), imiquimod, FSL-1, or loxoribin. The immunostimulatory ODN may be a CpG ODN. The TLR agonist may target TLR1, TLR2, TLR6, TLR7, TLR9, or TLR10.

As used herein, “a” or “an” may mean one or more. As used herein in the claim(s), when used in conjunction with the word “comprising”, the words “a” or “an” may mean one or more than one. As used herein “another” may mean at least a second or more. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The terms “inhibiting,” “reducing,” or “prevention,” or any variation of these terms, when used in the claims and/or the specification includes any measurable decrease or complete inhibition to achieve a desired result.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B—IL-21 receptor is expressed on B-CLL cells and is upregulated by CpG ODN. PBMC from 9 subjects with B-CLL were isolated, suspended in AIM-V medium and cultured for 2 hours, 4 days, or 7 days in the presence or absence of CpG ODN or control ODN at 2.5 μg/ml. Gene expression of the IL-21 receptor (n=9) was assessed with two different probes after 2 hours (FIG. 1A), IL-21 receptor protein expression on B-CLL cells (n=3) was measured by flow cytometry (median fluorescence intensity, MFI) on days 4 and 7 (FIG. 1B). Error bars indicate SEM.

FIGS. 2A-2B—Incubation with IL-21 and CpG ODN enhances survival of normal B cells, but decreases survival of CLL cells in vitro. (FIG. 2A) Impact of IL-21 on CLL cell survival. (FIG. 2B) Impact of IL-21 on survival of normal peripheral blood B cells.

FIGS. 3A-3B—CpG ODN and IL-21 are synergistic in their ability to induce apoptosis of CLL cells. (FIG. 3A) Isobologram of IL-21 and CpG ODN effect on CLL cells. (FIG. 3B) Effect of increasing concentrations of IL-21 and CpG ODN alone and together in inducing apoptosis of CLL cells.

FIGS. 4A-4B—CpG ODN and IL-21 induces apoptosis of purified CLL cells. (FIG. 4A) Two color histogram showing 99.9% of cells are CD5 positive and that CpG ODN plus IL21 induces apoptosis of CLL with both unfractionated cells and purified CLL cells. (FIG. 4B) CLL cells treated with CpG ODN and IL-21 are able to kill other CLL cells from the same donor that were not exposed to CpG ODN and IL-21 demonstrating cytotoxic effects against bystander cells. No such bystander effect was observed with no treatment, CpG ODN alone or IL-21 alone.

FIGS. 5A-5B—CpG ODN and IL21 selectively induce apoptosis of benign CD5(+) B cells from umbilical cord blood. (FIG. 5A) Ratio of CD5(+) to CD5(−) cells remaining after incubation of cells with IL-2 or IL-21 with or without CpG ODN. (FIG. 5B) Absolute number of CD5(+) cells remaining after incubation of cells with IL-2 or IL-21 with or without CpG ODN.

FIGS. 6A-6B—IL-21 plus CpG ODN induce apoptosis of B-CLL cells. PBMC from 9 subjects with B-CLL were cultured for 4 days in the presence of CpG ODN (2.5 μg/ml) and IL-21 (100 ng/ml) or IL-2 (100 U/ml). Cell survival of B-CLL cells was determined using Annexin V and PI staining and counterstaining with antibodies to CD19. (FIG. 6A) Shown are Annexin V/PI dot plots from one representative experiment. Gated are CD19+B-CLL cells. (FIG. 6B) The mean B-CLL cell survival rates from 9 independent experiments are plotted. Error bars indicate SEM.

FIGS. 7A-7B—The pro-apoptotic effect of IL-21 and CpG ODN on B-CLL cells is synergistic. PBMC from 3 subjects with B-CLL were cultured with CpG ODN and IL-21 at different concentration ratios for 4 days and the percentage of apoptotic cells was determined. (FIG. 7A) One representative experiment out of three demonstrating a synergistic interaction between CpG ODN and IL-21. A horizontal line indicates an additive interaction while the observed convex curve demonstrates synergy. (FIG. 7B) The effect of varying concentrations of CpG ODN and IL-21 demonstrated that both agents induced greater apoptosis than either agent alone, even at concentrations that give peak effects for each individual agent. Plotted are the mean cell survival rates (n=3). Error bars indicate SEM.

FIGS. 8A-8B—Pro-apoptotic effect of IL-21 and CpG ODN on B-CLL cells is direct. PBMC from 3 subjects with B-CLL were isolated and divided into two fractions (FIG. 8A=unpurified and FIG. 8B=purified). One fraction was purified to a percentage of >99% CD5(+), CD19(+) B-CLL cells. Both fractions were incubated for 3 days with IL-21, CpG ODN, or both agents and apoptosis was determined flowcytometrically. Dot plots demonstrate the purity of B cell populations based on CD19 expression. Bar graphs illustrate the mean B-CLL cell survival rates in response to treatment. Error bars indicate SEM.

FIGS. 9A-9C—IL-21 induces Granzyme B secretion by B-CLL cells, which is synergistically enhanced by CpG ODN. (FIG. 9A) PBMC from 5 subjects with B-CLL were isolated and cultured for 18 hours in the presence of different combinations of IL-21 (100 ng/ml), IL-2 (100 U/ml), and CpG ODN (2.5 μg/ml). For the last 4 hours, Brefeldin A at 1 μg/ml was added to the cells. Then cells were fixed, permeabilized and stained with antibodies to Perforin, Granzyme A and B or with control antibodies. One representative experiment out of 5 is shown. Gated are CD19+B-CLL cells. The dot plots show the percentages of Granzyme B+B-CLL cells in the presence or absence of IL-21 and CpG ODN. No expression of Granzyme A or Perforin could be detected (data not shown). Granzyme B expression in B-CLL cells was not induced by IL-2 (data not shown). (FIG. 9B) B-CLL cells from 3 subjects were isolated and purified to a percentage of at least 99.9% based on CD19 expression. The cells were then cultured on 96-well ELISpot plates for Granzyme B detection at the indicated cell number per well and in the presence of different B cell activators alone or with IL-21. After 16 hours plates were developed and dots counted. Every condition was run in triplicates. The average spot numbers from one representative experiment out of 3 are depicted. Error bars indicate standard deviation. (FIG. 9C) B-CLL cell survival after 4 days of incubation with anti-BCR or CpG ODN alone or with IL-21 was flow-cytometrically detected using Annexin V, anti-CD19, and PI staining. Averages from 3 independent experiments are shown. Error bars indicate SEM.

FIGS. 10A-10B—B-CLL cells treated with IL-21 plus CpG ODN can induce apoptosis of untreated, bystander B-CLL cells. Anti-Granzyme B antibodies inhibit bystander B-CLL cell killing. (FIG. 10A) Purified B-CLL cells were split into two fractions. One fraction was stained with PKH-26, then incubated for 24 hours in IL-21 with or without CpG ODN. Unstained cells were maintained in culture without stimulus. The stained, treated cells were washed, added to the untreated cells, and co-cultured for 2 days. Survival of the untreated cells (as indicated by lack of membrane dye) was analyzed by flow cytometry. Plotted are the mean B-CLL cell survival rates for the untreated (i.e., bystander) cells cultured at different ratios with treated cells. One representative experiment out of three with similar results is shown. (FIG. 10B) B-CLL cells from 3 subjects were cultured for 4 days in the presence of IL-21 (100 ng/ml), CpG ODN (2.5 μg/ml), and anti-human Granzyme B antibody at 10 and 20 μg/ml or a control antibody at 20 μg/ml. B-CLL cell survival was determined by FACS analysis using Annexin V/PI staining and counterstaining with antibodies to CD19. Plotted are the mean B-CLL cell survival rates in percent. Error bars indicate SEM.

FIGS. 11A-11C—IL-21 induces Granzyme B secretion by benign peripheral B cells. (FIG. 11A) PBMC from healthy subjects were isolated, B cells purified (>99.9%) and cultured for 20 hours in the presence of various B cell stimulators alone or with IL-21 (100 ng/ml). For the last 4 hours Brefeldin A at 1 μg/ml was added to the cells. Then cells were fixed, permeabilized and stained with antibodies to CD27 and Perforin, Granzyme B or isotype control. One representative experiment out of 6 is shown. All cells are positive for CD19. The dot plots show the percentages of Granzyme B positive (+) B cells. No expression of Perforin could be detected (data not shown). (FIG. 11B) PBMC from 7 subjects were isolated and B cells purified to a percentage of at least 99.5% based on CD19 expression. The B cells were then cultured on 96-well ELISpot plates for Granzyme B detection (100,000 cells per well) in the presence of different B cell activators alone or with IL-21. After 16 hours plates were developed and dots counted. Every condition was run in triplicates. The average spot numbers from one representative experiment out of 7 are depicted. Error bars indicate standard deviation (SD). (FIG. 11C) PBMC from 4 healthy subjects were isolated, B cells purified (99.9%) and cultured for 3 days in the presence of various B cell stimulators alone or in combination with IL-21 (100 ng/ml). Cell survival of B cells was determined using Annexin V and PI staining and counterstaining with antibodies to CD19. The mean B cell survival rates from one representative experiment out of 3 with similar results are plotted. Error bars indicate SD.

FIG. 12—DNA fragmentation in B-CLL cells is detectable as early as 12 hours after start of incubation with IL-21 and CpG ODN. B-CLL cells were isolated, purified to a percentage of at least 99.9% based on CD19 staining and cultured in the presence of IL-21 and CpG ODN for 12 hours. Then cells were harvested, fixed, permeabilized and FITC-labeled dUTP was enzymatically linked to fragmented DNA. Histograms show the percentages of B-CLL cells positive for dUTP.

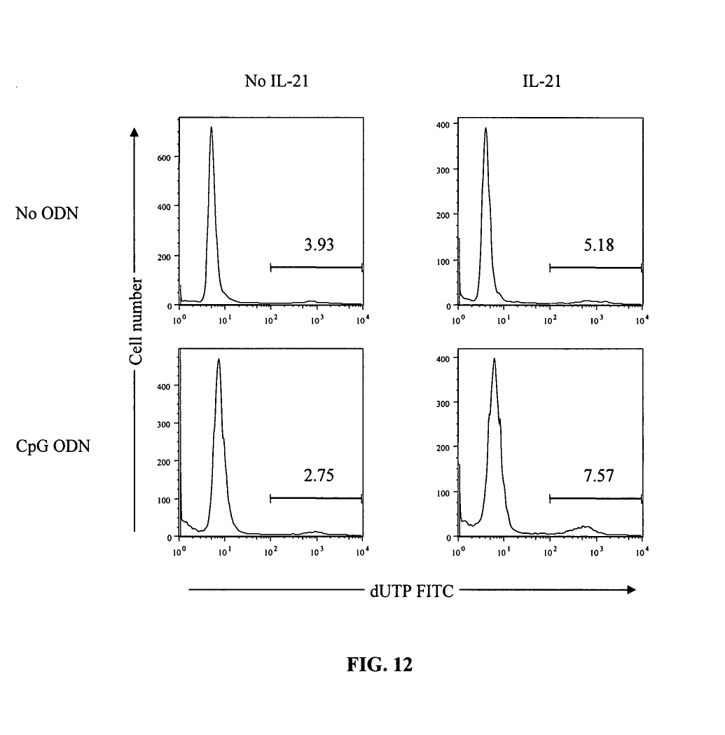

FIG. 13—IL-21 and CpG ODN induce B-CLL cell expression of lysosome-associated molecular protein 1 (LAMP-1, CD107a). PBMC from 7 subjects with B-CLL were cultured for 4 days in the presence of CpG ODN (2.5 μg/ml) and IL-21 (100 ng/ml). Expression of CD107a (LAMP-1) on CD19+B-CLL cells was determined using FACS analysis. Plotted are the relative median fluorescence intensities (MFI) for CD107a expression as compared to unstimulated cells. Error bars indicate SEM.

FIGS. 14A-14C—B-CLL cells secrete Granzyme B in response to IL-21. B-CLL cells were purified based on CD19 expression to a percentage of >99.9%. The cells were then cultured at 37° C. on 96-well EliSpots plates for Granzyme B detection at the indicated cell number per well and in the presence of different B cell activators alone or with IL-21. After 16 hours plates were developed and dots counted. Every condition was run in triplicates. FIG. 14A shows one representative experiment with decreasing cell numbers of purified B-CLL cells. FIG. 14B shows PHA controls with whole PBMC and purified B-CLL cells. FIG. 14C shows the effect of different B cell activators on IL-21-induced Granzyme B secretion. Individual plates of two representative experiments out of 3 are shown.

FIGS. 15A-15B—Granzyme B produced by B-CLL cells is able to cleave specific substrate and is accompanied by the occurrence of functionally active Caspase 6. PBMC from 2 subjects with B-CLL were isolated and cultured for 4 days in the presence of IL-21 (100 ng/ml) and CpG ODN (2.5 μg/ml). Activity of Granzyme B and Caspase 6 in B-CLL cells was determined using specific cell-permeable fluorogenic substrates and staining of CD19. The percentages of B-CLL cells positive for activated Granzyme B (FIG. 15A) and Caspase 6 (FIG. 15B) are shown, averaged from two independent experiments.

FIGS. 16A-16C—Benign B cells secrete Granzyme B in response to IL-21. Healthy peripheral B cells were purified based on CD19 expression to a percentage of >99.5%. The cells were then cultured at 37° C. on 96-well EliSpots plates for Granzyme B detection at the indicated cell number per well and in the presence of different B cell activators alone or with IL-2 or IL-21. After 16 hours plates were developed and dots counted. Every condition was run in triplicates. FIG. 16A shows one representative experiment with different B cell activators. FIG. 16B shows PHA controls with whole PBMC and purified B cells. FIG. 16C shows the effect of IL-2 and IL-21 in the presence or absence of CpG ODN on Granzyme B secretion by B cells. Individual plates of two representative experiments out of 7 are shown.

FIG. 17—Interleukin 21 (IL-21) induces de-novo synthesis of granzyme B in benign human peripheral B cells. Peripheral blood mononuclear cells (PBMC) from healthy donors were incubated for 18 hours in the presence of IL-21, CpG ODN, anti-CD40 antibodies, anti-BCR, or combinations. Then Brefeldin A was added and after four hours cells were harvested, fixed, permeabilized, stained with fluorescently-labeled antibodies to CD19 and granzyme B and analyzed flow cytometrically.

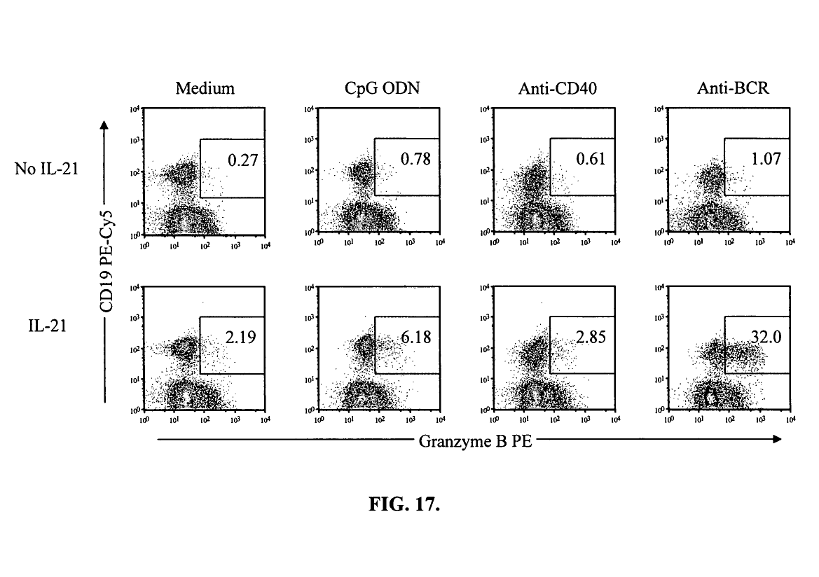

FIG. 18—IL-21 is not the only cytokine capable of inducing granzyme B in B cells. B cells were purified as described (purity >99.9%) and incubated in the presence of anti-BCR and various cytokines or cytokine combinations on granzyme B ELISpot plates. After 16 hours plates were developed and granzyme B secretion analyzed on an ELISpot reader. New combinations apart from IL-21 that induce granzyme B in B cells include IL-4 and IL-10, and IL-10 and IFNα.

FIG. 19—Bone marrow stroma cells can induce granzyme B secretion by pre-activated B cells in the absence of IL-21. Highly purified B cells (purity >99.9%) were incubated for 16 hours on granzyme B ELISpot plates in the presence of IL-21, CpG ODN, B cell receptor stimulation or various combinations at 10⁵ cells/well (upper row). Alternatively IL-21 was replaced by 15×10³ bone marrow stroma cells (HS-5) (intermediate row). As a control stroma cells were also incubated alone (lower row).

FIG. 20—IL-21 induces transcription of the genes for granyzme B and perforin in B cells. Purified B cells (purity >99.9%) were cultured for 18 hours in the presence or absence of IL-21 and B cell receptor stimulation (anti-BCR antibodies). Subsequently RNA was isolated and real time RT-PCR with specific primers and probes for granzyme B and perforin was performed. In addition to real time RT-PCR, conventional RT-PCR was also performed and PCR products were run on a gel. The expected amplicon sized are 218 bp (base pairs) and 146 bp for granzyme B and perforin respectively. The table shows the number of specifically detected mRNA copies per μg total RNA.

FIG. 21—IL-21 induces the secretion of interferon-γ (IFN-γ) by daudi cells. Daudi cells (pre-B cell line) were incubated on IFN-γ-ELISpot plates in the presence of IL-21, CpG ODN, B cell receptor stimulation or various combinations at a cell concentration of 10⁵ cells/well. After 16 hours plates were developed and analyzed.

FIG. 22—B cells can inhibit the expansion of allogeneic CD4+T cells in the presence of IL-21, B cell receptor stimulation and CpG ODN. Highly purified B cells and allogeneic CD4+T cells were co-incubated in the presence of IL-21, B cell receptor stimulation and CpG ODN for 5 days. Then the number of viable CD4+T cells was detected flow cytometrically using Annexin V and PI.

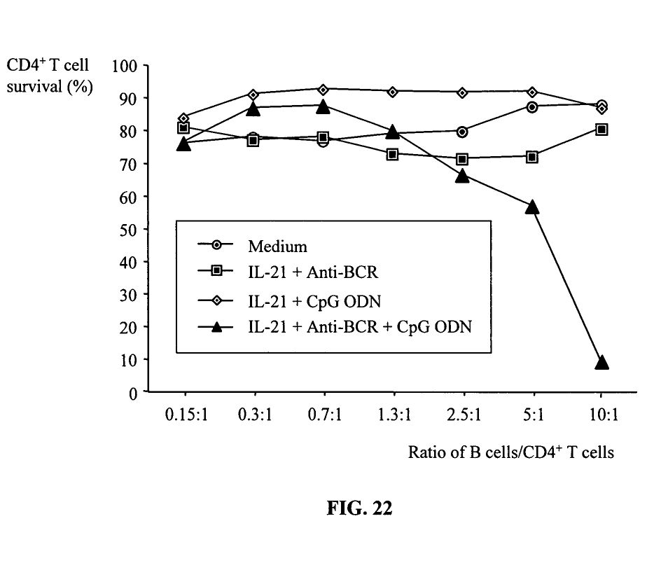

FIG. 23—Principle for the differentiation of human B cells into cytotoxic B lymphocytes (CBL). A local bacterial infection activates B cells via TLR9 and the B cell receptor. Simultaneously activated CD4+T cells secrete IL-21, which induces the differentiation of pre-activated B cells to granzyme B-secreting cytotoxic B lymphocytes (left side). In contrast, if ligation of CD40 by CD4+T cells overweighs TLR9 and B cell receptor signaling, B cells differentiate into antibody-producing plasma cells (right side).

### DETAILED DESCRIPTION OF THE INVENTION

As discussed above, there is a great need for improved methods of treating cancers and autoimmune disorders, particularly those that are associated with pathologic immunoglobulin production. The present invention addresses these disease states by providing a new therapeutic intervention that uses a combination of the cytokine IL-21 and a toll-like receptor agonist to inhibit cytokine production, B cell growth, B cell division or antibody production, including both B1 and B2 cell subsets. This therapy can be combined with more traditional interventions, such as chemotherapy and radiotherapy for cancers, and anti-inflammatory and immunosuppressive therapies for immune disease. The specifics of the invention are discussed below.

### I. INTERLEUKIN 21 (IL-21)

IL-21 is a cytokine of 131 residues (SEQ ID NO:1) and characterized by a four-helix bundle and sequence homologies to IL-2 and IL-15. It is known to mediate its biological action via the IL-21R, composed of a specific chain, IL-21Rα, and the common γ-chain (CD132). Recent data suggest that IL-21 possesses pro-inflammatory properties. The IL-21 receptor (IL-21R) is expressed in lymphoid tissue, in particular by NK, B, T and dendritic cells, macrophages and endothelial cells. It is a key factor in the transition between innate and adaptive immune responses secreted by activated T cells. Recent evidence suggests that IL-21 plays a supportive role in the proliferation of T and B cells and influences the cytolytic activity of natural killer cells. IL-21 has been shown to up-regulate genes associated with innate immunity and cytotoxicity including Granzyme A and B and to inhibit the differentiation of naïve T helper cells.

IL-21 specifically inhibits IFN-γ production from developing TH1 cells and is preferentially expressed by TH2 cells. Furthermore IL-21 has been identified as a growth and survival factor for human myeloma cells. IL-21/IL-21R interactions have a unique role in sequentially activating both innate and adaptive immune responses against poorly immunogenic tumors, leading to tumor rejection that is perforin dependent but IFN-γ independent. It has been proposed that IL-21 attracts neutrophils indirectly in vivo via a mechanism independent of IL-6, CCL3, CCL5, and CXCL2 production.

### II. TOLL LIKE RECEPTOR (TLR) AGONISTS

In accordance with the present invention, IL-21 is provided to a subject in conjunction with a TLR agonist. The following is an exemplary but non-limiting list of TLR agonists that may be used in conjunction with the present invention.

A. Immunostimulatory ODNs

In one embodiment, the TLR agonists maybe immunostimulatory oligonucleotides (ODNs). U.S. Pat. Nos. 6,821,957, 6,667,293, 6,610,661, 6,589,940, 6,562,798, 6,558,670, 6,544,518, 6,498,148, 6,406,705, 6,339,068, 6,218,371 and 5,968,909 describe immunostimulatory ODNs, and in particular CpG ODNs.

B. Imiquimod

Imiquimod is an immune response modifier. It is marketed as a 5% cream called Aldara™. Imiquimod is mainly used to treat genital warts, solar keratoses, and basal cell skin cancers. Imiquimod works by stimulating the immune system to release a number cytokines. When used to treat skin cancers and pre-cancerous lesions it results in inflammation, which destroys the lesion. The degree of inflammation is quite variable from person to person, in part due to the type of skin lesion and in part due to genetic factors. Imiquimod is binding intracellularly to TLR7. Imiquimod is particularly useful on areas where surgery or other treatments may be difficult, complicated or otherwise undesirable, especially the face and lower legs. A course of treatment ranges from 4 to 16 weeks.

C. FSL-1

The lipopeptide FSL-1 [S-(2,3-bispalmitoyloxypropyl)-Cys-Gly-Asp-Pro-Lys-His-Pro-Lys-Ser-Phe, Pam(2)CGDPKHPKSF (SEQ ID NO:3)] is an agonist for TLRs 2 and 6 and is able to activate NF-kappa B, comparable to ligands of other TLRs. There are no clinical applications so far.

D. Loxoribine

Loxoribine (7-allyl-8-oxoguanosine) is a TLR7 agonist, with biologic effects on B cells comparable to those seen with other TLR7 and TLR9 ligands. Loxoribine has shown anti-tumor activity and enhancement of NK cell activity in vitro. There are no clinical applications so far.

### III. GRANZYME B

A key killing mechanism used by cytotoxic T lymphocytes (CTL) and natural killer (NK) cells is the release of cytotoxic granules into the secretory synapse between effector and target cell (Bossi et al., 2002). A major constituent of these granules is the 32-kDa serine protease Granzyme B (Wowk and Trapani, 2004; Trapani and Sutton, 2003) (also known as cytotoxic T-lymphocyte-associated serine esterase 1 (CTLA1), Granzyme 2, protease serine B (CSPB) and cathepsin G-Like 1 (CGL1)). Granzyme B is activated by Cathepsin C after its release into the secretory synapse, taken up into the target cell via fluid phase and mannose-6-phosphate receptor-mediated endocytosis (Wowk and Trapani, 2004), and released from the endosome in response to a second signal, such as Perforin, or microbial factors such as adenovirus or certain bacterial toxins (Lord et al., 2003; Forelich et al., 1996; Browne et al., 1999; Russell and Ley, 2002). The transduction of the death signal mediated by Granzyme B in the target cell occurs via two pathways, one by direct signaling to the mitochondria via BID and one by activation of the classical caspase cascade. Granzyme B is one of the most effective and fastest executioners of apoptosis known. To date, CTL and NK cells are the only cell populations known to express and secrete Granzyme B.

In a differential cDNA bank, Brunet et al. (1986) detected 3 distinct mRNA transcripts (CTLA1, CTLA2, and CTLA3) in various cytotoxic T cells but not (or less so) in a range of non-cytotoxic lymphoid cells. They described the co-inducibility of these transcripts, the sequence of CTLA1 cDNA, and its protein homology with serine esterases. Klein et al. (1989) isolated and sequenced the cytotoxic serine protease B gene. Due to faulty or at least variable intron/exon splicing, the mRNA transcripts from the human serine protease gene are heterogeneous in size. Two cryptic splice sites, used to generate these aberrant mRNA transcripts, were identified. Hanson et al. (1990) used a cathepsin G cDNA probe (see NCBI protein database GI:116830) to clone 2 cathepsin G-like genes (designated CGL1 and CGL2) from a human genomic library. They determined that CGL1 is identical to the previously identified gene CTLA1, or serine protease B, that is expressed only in activated cytotoxic T lymphocytes. Dahl et al. (1990) isolated cDNA clones from a human NK cell cDNA library that encode granzyme B. They suggested that the granzyme B gene is homologous to that for CTLA1. Rissoan et al. (2002) detected granzyme B mRNA in both resting and activated plasmacytoid dendritic cells and at much lower levels in monocytes, resting T cells, B cells, activated granulocytes, and activated monocyte-derived dendritic cells. It was barely detectable in nonactivated monocyte-derived dendritic cells.

Klein et al. (1989) found that the cytotoxic serine protease B gene is approximately 3,500 bp long, consisting of 5 exons and 4 introns. Haddad et al. (1990) reported that the CTLA1 gene is about 4.75 kb long. Brunet et al. (1986) found by in situ hybridization that the Ctla1 gene maps to the D segment of mouse chromosome 14, where the Tcra gene (see Online Medelian Inheritance in Man (OMIM) database 186880) is also situated. Preliminary experiments suggested that the human gene might also be situated close to TCRA. Such was confirmed by Harper et al., (1988). In addition to in situ hybridization, linkage analysis was performed using interspecific mouse backcrosses; no recombination was observed in 100 backcross products studied. Using DNA blot analysis on a panel of human-rodent somatic cell lines, Klein et al. (1989) localized the CSPB gene to chromosome 14. Using a human cell line with an inversion on chromosome 14, Harper et al. (1988) showed that the order of loci on 14q is NP (164050)—TCRA—CTLA1. Two components of the complement cascade that possess serine protease domains, namely, C2 and factor B, map close to the MHC class I and class II loci. The serine esterase trypsin gene maps close to the TCRB locus. The close proximity of TCRA and CTLA1 provides another example of the proximity of genes coding for a member of the Ig superfamily and a serine esterase. Hanson et al. (1990) showed that cathepsin G (116830), CGL1, and CGL2 (116831) are linked on a 50-kb segment in band 14q11.2. Thus, this gene cluster maps to the same chromosomal band as the alpha and delta T cell receptor gene—a region involved in most chromosomal translocations and inversions specifically associated with T cell malignancies. For the physical linkage studies of the 3 genes, Hanson et al. (1990) screened a human cosmid library with probes for all 3 genes. In this way they found that CGL1 and CGL2 are separated by about 21 kb, and that the cathepsin G gene is about 31 kb downstream of CGL2. The 3 genes are in the same 5-prime to 3-prime orientation. The murine homolog of CGL1 has been mapped to mouse chromosome 14 (Crosby et al., 1990). Band 14q11.2 contains a cluster of genes involved in hematopoietic development: CGL1 in activated cytotoxic lymphocytes, cathepsin G in promyelocytes and promonocytes and the alpha/delta T cell receptor genes in early T cell ontogeny. Lin et al. (1990) also assigned CTLA1, as well as another serine protease gene, to 14q11.2-q12 by in situ hybridization. By Southern analysis of rodent-human hybrid cells retaining various chromosome 14 rearrangements, Dahl et al. (1990) localized the gene to 14q11-q32, distal to the T cell receptor alpha locus and proximal to the immunoglobulin heavy chain locus.

### IV. PROTEIN PURIFICATION

It may be desirable as part of the present invention to purify IL-21. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; and/or isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins; other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical, and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### V. NUCLEIC ACIDS

In various embodiments, one may wish to produce a cytokine such as IL-21 or IL-10 in a recombinant fashion. For example, a nucleic acid encoding IL-21 may be inserted into an expression vector for using in producing IL-21 that is purified for subsequent administration to a patient as discussed above. Alternatively, the vector may itself be administered to a subject followed by expression of IL-21 in an appropriate target cell.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. In accordance with the present invention, the DNA sequence of IL-21 is provided in SEQ ID NO:2.

A. Preparation of Nucleic Acids

A nucleic acid may be prepared by any technique known to one of ordinary skill in the art, such as for example, chemical synthesis, enzymatic production, or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, or 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference). In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, the bulk of cellular components or in vitro reaction components such as for example, macromolecules such as lipids or proteins, small biological molecules, and the like.

C. Vectors for Cloning, Gene Transfer and Expression

Within certain embodiments, expression vectors are employed to express IL-21 or other therapeutic genes, antisense constructs, ribozymes or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

1. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et al.; |
| | 1990 |
| Immunoglobulin Light Chain | Queen et al., 1983; Picard et al., 1984 |
| T Cell Receptor | Luria et al., 1987; Winoto et al., 1989; Redondo et al.; 1990 |
| HLA DQ a and/or DQ β | Sullivan et al., 1987 |
| β-Interferon | Goodbourn et al., 1986; Fujita et al., 1987; Goodbourn et al., |
| | 1988 |
| lnterleukin-2 | Greene et al., 1989 |
| Interleukin-2 Receptor | Greene et al., 1989; Lin et al., 1990 |
| MHC Class II 5 | Koch etat., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., 1989 |
| Prealbumin (Transthyretin) | Costa et al., 1988 |
| Elastase I | Ornitzetat., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., 1985; |
| | Firak et al., 1986; Herr et al., 1986; Imbra et al., 1986; |
| | Kadesch et al., 1986; Wang et al., 1986; Ondek et al., 1987; |
| | Kuhl et al., 1987; Schaffner et al., 1988 |
| Polyoma | Swartzendruber et al., 1975; Vasseur et al., 1980; Katinka et |
| | al., 1980, 1981; Tyndell et al., 1981; Dandolo et al., 1983; de |
| | Villiers et al., 1984; Hen et al., 1986; Satake et al., 1988; |
| | Campbell and/or Villarreal, 1988 |
| Retroviruses | Kriegler et al., 1982, 1983; Levinson et al., 1982; Kriegler et |
| | al., 1983, 1984a, b, 1988; Bosze et al., 1986; Miksicek et al., |
| | 1986; Celander et al., 1987; Thiesen et al., 1988; Celander et |
| | al., 1988; Choi et al., 1988; Reisman et al., 1989 |
| Papilloma Virus | Campo et al., 1983; Lusky et al., 1983; Spandidos and/or |
| | Wilkie, 1983; Spalholz et al., 1985; Lusky et al., 1986; Cripe |
| | et al., 1987; Gloss et al., 1987; Hirochika et al., 1987; |
| | Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et al., |
| | 1988; Feng et al., 1988; Takebe et al., 1988; Rosen et al., |
| | 1988; Berkhout et al., 1989; Laspia et al., 1989; Sharp et al., |
| | 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester | Palmiter et al., 1982; |
| | (TFA) | Haslinger et al., 1985; |
| | Heavy metals | Searle et al., 1985; |
| | | Stuart et al., 1985; |
| | | Imagawa et al., 1987, |
| | | Karin et al., 1987; |
| | | Angel et al., 1987b; |
| | | McNeall et al., 1989 |
| MMTV (mouse | Glucocorticoids | Huang et al., 1981; |
| mammary tumor | | Lee et al., 1981; |
| virus) | | Majors et al., 1983; |
| | | Chandler et al., 1983; |
| | | Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large | Taylor et al, 1989, |
| | T Antigen | 1990a, 1990b |
| Proliferin | Phorbol | Mordacq et al., 1989 |
| | Ester-TPA | |
| Tumor Necrosis | PMA | Hensel et al., 1989 |
| Factor | | |
| Thyroid | Thyroid | Chatterjee et al., |
| Stimulating | Hormone | 1989 |
| Hormone α Gene | | |

Of particular interest are promoters that are selectively active in B-cells. A particular promoter in this group is the CD19 promoter (Maas et al., 1999).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

2. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

3. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap-dependent translation and begin translation at internal sites (Pelletier and Sonenberg, 1988). IRES elements from two members of the picarnovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

4. Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Epstein-Barr virus, frequently referred to as EBV, is a member of the herpesvirus family and one of the most common human viruses. The virus occurs worldwide, and most people become infected with EBV sometime during their lives. In the United States, as many as 95% of adults between 35 and 40 years of age have been infected. When infection with EBV occurs during adolescence or young adulthood, it causes infectious mononucleosis 35% to 50% of the time. EBV vectors have been used to efficiently deliver DNA sequences to cells, in particular, to B lymphocytes. Robertson et al. (1986) provides a review of EBV as a gene therapy vector.

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell, the nucleic acid encoding the gene of interest may be positioned and expressed at different sites. In certain embodiments (e.g., recombinant expression in vitro), the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085). In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, antibodies to CD5 (CLL) and CD22 (lymphoma) can be used as targeting moieties.

### VI. PHARMACEUTICAL FORMULATIONS AND ROUTES OF ADMINISTRATION

The present invention also involves the provision of therapeutic amounts of IL-21 and TLR agonists for the treatment of cancer and immune diseases. The types of cancer that may be treated, according to the present invention, are B1 B cell related cancers. Treatment does not require that the tumor cell be killed or induced to undergo normal cell death or “apoptosis.” Rather, to accomplish a meaningful treatment, the tumor cell growth may simply be slowed to some degree, or the effects of the B cell dysregulations (secretion of cytokines or antibodies) be reducted. Clinical terminology such as “remission” and “reduction of tumor cell burden” also are contemplated given their normal usage. In fact, any clinical benefit fulfills the term “treatment.” Similarly, with regard to immune diseases, treatment may encompass B cell killing or apoptosis, but may also include reduction in antibody or cytokine production.

In one embodiment, the present invention provides for a combination therapy of IL-21 and a TLR agonist. The two agents may therefore be formulated together. However, separate administration, either at the same time or at distinct times may be employed. Another therapeutic embodiment contemplated by the present inventors is to utilize an expression construct expressing IL-21 in a cell, which is provided in conjunction with a TLR agonist. The lengthy discussion of expression vectors and the genetic elements employed therein is incorporated into this section by reference.

Various routes are contemplated for various disease states types. Systemic delivery, such as intravenous, subcutaneous or intraarterial delivery, is contemplated. This will prove especially important for attacking microscopic or metastatic cancer or immune disease. Where a discrete tumor mass may be identified, a variety of direct, local and regional approaches (lymphatic) may be taken. For example, the tumor may be directly injected with IL-21/expression vector and the TLR agonist. A tumor bed may be treated prior to, during or after resection. Following resection, one generally will deliver the therapeutic by a catheter left in place following surgery.

In a different embodiment, ex vivo therapy is contemplated. In an ex vivo embodiment, cells from the patient are removed and maintained outside the body for at least some period of time. During this period, the therapy is delivered, after which the cells are reintroduced into the patient; hopefully, any undesireable cells in the sample have been killed or otherwise inhibited.

Pharmacological therapeutic agents and methods of administration, dosages, etc., are well known to those of skill in the art (see for example, the “Physicians Desk Reference,” Goodman and Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage of the agents will necessarily occur depending on the condition of the subject being treated. The person responsible for administration will, in any event, determine the appropriate dose for the individual subject, and such invidual determinations are within the skill of those of ordinary skill in the art.

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render the agents stable. Aqueous compositions of the present invention comprise an effective amount of the agents, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the compositions.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108, 4,166,452, and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the the partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For topical use, creams, ointments, jellies, gels, epidermal solutions or suspensions, etc., containing a therapeutic compound are employed. For purposes of this application, topical application shall include mouthwashes and gargles. Formulations may also be administered as nanoparticles, liposomes, granules, inhalants, nasal solutions, or intravenous admixtures.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### VII. B CELL IMMUNE DISEASES

A. Autoimmune Diseases and Diseases of Excessive Immunoglobulin Production

Most autoimmune diseases have in common that B1 cell levels are elevated. Although the pathogentic role of this is still not clearly understood, B1 cells seem to be necessary to initiate and/or perpetuate the autoimmune process. The ability to eliminate B1 cells could impact on each of these disease states.

1. Systemic Lupus Erythematosus (SLE)

SLE is an autoimmune rheumatic disease characterized by deposition in tissues of autoantibodies and immune complexes leading to tissue injury (Kotzin, 1996). In contrast to autoimmune diseases such as MS and type 1 diabetes mellitus, SLE potentially involves multiple organ systems directly, and its clinical manifestations are diverse and variable (Reviewed by Kotzin and O'Dell, 1995). For example, some patients may demonstrate primarily skin rash and joint pain, show spontaneous remissions, and require little medication. At the other end of the spectrum are patients who demonstrate severe and progressive kidney involvement that requires therapy with high doses of steroids and cytotoxic drugs such as cyclophosphamide (Kotzin, 1996). More recently, treatment with rituximab (anti-CD2) has been attempted.

The serological hallmark of SLE, and the primary diagnostic test available, is elevated serum levels of IgG antibodies to constituents of the cell nucleus, such as double-stranded DNA (dsDNA), single-stranded DNA (ss-DNA), and chromatin. Among these autoantibodies, IgG anti-dsDNA antibodies play a major role in the development of lupus glomerulonephritis (Hahn and Tsao, 1993; Ohnishi et al., 1994). Glomerulonephritis is a serious condition in which the capillary walls of the kidney's blood purifying glomeruli become thickened by accretions on the epithelial side of glomerular basement membranes. The disease is often chronic and progressive and may lead to eventual renal failure.

The mechanisms by which autoantibodies are induced in these autoimmune diseases remains unclear. As there has been no known cause of SLE, to which diagnosis and/or treatment could be directed, treatment has been directed to suppressing immune responses, for example with macrolide antibiotics, rather than to an underlying cause. (e.g., U.S. Pat. No. 4,843,092).

2. Rheumatoid Arthritis (RA)

The exact etiology of RA remains unknown, but the first signs of joint disease appear in the synovial lining layer, with proliferation of synovial fibroblasts and their attachment to the articular surface at the joint margin (Lipsky,1998). Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumor necrosis factor (TNF-α), which plays a role in inflammation (Dinarello, 1998; Arend and Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

In normal joints, the effects of these and other proinflammatory cytokines are balanced by a variety of anti-inflammatory cytokines and regulatory factors (Burger and Dayer, 1995). The significance of this cytokine balance is illustrated in juvenile RA patients, who have cyclical increases in fever throughout the day (Prieur et al., 1987). After each peak in fever, a factor that blocks the effects of IL-1 is found in serum and urine. This factor has been isolated, cloned and identified as IL-1 receptor antagonist (IL-1Ra), a member of the IL-1 gene family (Hannum et al., 1990). IL-IRa, as its name indicates, is a natural receptor antagonist that competes with IL-1 for binding to type I IL-1 receptors and, as a result, blocks the effects of IL-1 (Arend et al., 1998). A 10- to 100-fold excess of IL-IRa may be needed to block IL-1 effectively; however, synovial cells isolated from patients with RA do not appear to produce enough IL-1Ra to counteract the effects of IL-1 (Firestein et al., 1994; Fujikawa et al., 1995).

3. Systemic Sclerosis

Systemic sclerosis (SSc) is a connective tissue disease of unknown etiology characterized by fibrotic changes of the skin, subcutaneous tissue, and viscera; abnormalities of the microvasculature; and immune dysfunction. The literature has referred to the skin changes as “keloid” in nature. In early national surveys from the ‘60’s, the incidence of SSc was reported to be 12 cases per 1 million population annually. More recent studies report a higher prevalence of SSc, on the order of 19-75 case per 100,000 population.

SSc can affect a wide variety of organs and tissues including the skin, gastrointestinal tract, lungs, heart, kidneys, and musculoskeletal system. Altered connective tissue metabolism characterized by increased deposition of extracellular matrix components like collagen, fibronectin and glycosaminoglycans has been observed in SSc. Lymphokines such IL-2, IL-4, and IL-6 were found in the sera of patients with scleroderma, but not in healthy control subjects. Activated T cells and/or antibody-dependent complement cascade likely stimulate the release of endothelial cytokines with subsequent endothelial damage, which facilitates adhesion and migration T cells and monocytes.

4. Polymyositis

Polymyositis is an inflammatory muscle disease that causes varying degrees of decreased muscle power. The disease has a gradual onset and generally begins late in the second decade of life. The primary symptom is muscle weakness, usually affecting those muscles that are closest to the trunk of the body (proximal). Eventually, patients have difficulty rising from a sitting position, climbing stairs, lifting objects, or reaching overhead. In some cases, distal muscles (those not close to the trunk of the body) may also be affected later in the course of the disease. Trouble with swallowing (dysphagia) may occur. The disease may be associated with other collagen vascular, autoimmune or infectious disorders. Treatment for generally consists of prednisone or immunosuppressants such as azathioprine and methotrexate.

5. Sjögren's Syndrome

Sjögren's syndrome is a systemic autoimmune disease in which the body's immune system mistakenly attacks its own moisture producing glands. Sjögren's is one of the most prevalent autoimmune disorders, striking as many as 4,000,000 Americans, with 90% of patients being women. The average age of onset is late 40's although Sjögren's occurs in all age groups in both women and men.

About 50% of the time Sjögren's syndrome occurs alone, and 50% of the time it occurs in the presence of another connective tissue disease. The four most common diagnoses that co-exsist with Sjögren's syndrome are Rheumatoid Arthritis, Systemic Lupus, Systemic Sclerosis (scleroderma) and Polymyositis/Dermatomyositis. Sometimes researchers refer to these situations as “Secondary Sjögren's.”

Sjögren's is characterized by dry eyes and dry mouth, and may also cause dryness of other organs such as the kidneys, GI tract, blood vessels, lung, liver, pancreas, and the central nervous system. Many patients experience debilitating fatigue and joint pain. Symptoms can plateau, worsen, or go into remission. While some people experience mild symptoms, others suffer debilitating symptoms that greatly impair their quality of life.

6. Grave's Disease

Marked by nervousness and overstimulation, Grave's disease is the result of an overactive thyroid gland (hyperthyroidism). Thyroid hormones regulate metabolism and body temperature, and are essential for normal growth and fertility. But in excessive amounts, they can lead to the burn-out seen in this relatively common form of thyroid disease. It is unclear what triggers this problem, but the immune system is involved. In Grave's disease patients, they find antibodies specifically designed to stimulate the thyroid.

Along with nervousness and increased activity, Grave's disease patients may suffer a fast heartbeat, fatigue, moist skin, increased sensitivity to heat, shakiness, anxiety, increased appetite, weight loss, and sleep difficulties. They also have at least one of the following: an enlargement of the thyroid gland (goiter), bulging eyes, or raised areas of skin over the shins.

In many cases, drugs that reduce thyroid output are sufficient to control the condition. A short course of treatment with radioactive iodine, which dramatically reduces the activity of the thyroid, is another option for people past their childbearing years. In some cases, surgery to remove all or part of the thyroid (thyroidectomy) is needed. Surgery can also relieve some of the symptoms of Grave's disease. Bulging eyes, for example, can be corrected by creating enough extra space in the nearby sinus cavity to allow the eye to settle into a more normal position.

7. Myasthenia Gravis

The number of myasthenia gravis patient in the United States alone is estimated at 0.14% of the population, or approximately 36,000 cases; however, myasthenia gravis is likely under diagnosed. Previously, women appeared to be more often affected than men, with the most common age at onset being the second and third decades in women, and the seventh and eighth decades in men. As the population ages, the average age at onset has increased correspondingly, and now males are more often affected than females, and the onset of symptoms is usually after age 50.

Patients complain of specific muscle weakness and not of generalized fatigue. Ocular motor disturbances, ptosis or diplopia, are the initial symptom of myasthenia gravis in two-thirds of patients. Oropharyngeal muscle weakness, difficulty chewing, swallowing, or talking, is the initial symptom in one-sixth of patients, and limb weakness in only 10%. Initial weakness is rarely limited to single muscle groups such as neck or finger extensors or hip flexors. The severity of weakness fluctuates during the day, usually being least severe in the morning and worse as the day progresses, especially after prolonged use of affected muscles. The course of disease is variable but usually progressive, resulting in permanent muscle weakness. Factors that worsen myasthenic symptoms are emotional upset, systemic illness (especially viral respiratory infections), hypothyroidism or hyperthyroidism, pregnancy, the menstrual cycle, drugs affecting neuromuscular transmission, and increases in body temperature.

In acquired myasthenia gravis, post-synaptic muscle membranes are distorted and simplified, having lost their normal folded shape. The concentration of ACh receptors on the muscle end-plate membrane is reduced, and antibodies are attached to the membrane. ACh is released normally, but its effect on the post-synaptic membrane is reduced. The post-junctional membrane is less sensitive to applied ACh, and the probability that any nerve impulse will cause a muscle action potential is reduced.

8. Mononucleosis

Infectious mononucleosis, or “glandular fever,” is caused by the Epstein-Barr virus. Though usually not serious, splenic rupture is possible second to enlargement of the spleen. Like most herpesviruses, EBV will go latent in neural ganglia after ther active infective subsides. Some people with mono have minimal symptoms, such as fatigue, fever, sore throat and headache. Reports of chronic, sub-acute infection exist. One exposed, most people will develop immunity and will not be reinfected. One notable characteristic, and the basis for the disease name, is the presence of an elevated white blood cell count. In severe forms of the disease, hyper-IgM production is observed

9. Hyper-IgM Syndrome

Patients with X-linked hyper-IgM (XHIGM) syndrome have a defect or deficiency in CD40 ligand, a protein that is found on the surface of T-lymphocytes. CD40 ligand is made by a gene on the X chromosome. Thus, this primary immunodeficiency disease is inherited as an X-linked recessive trait, and usually found only in boys. As a consequence of their deficiency in CD40 ligand, affected patients' T-lymphocytes are unable to instruct B-lymphocytes to switch their production of gam-maglobulins from IgM to IgG and IgA. Patients with this primary immunodeficiency disease have decreased levels of serum IgG and IgA and normal or elevated levels of IgM. In addition, since CD40 ligand is important to other functions of T-lymphocytes, they also have a defect in some of the protective functions of their T-lymphocytes. Other forms of autosomal recessive Hyper-IgM syndrome have been discovered, but the responsible mutations have not yet been identified.

10. Hyper-IgD Syndrome

The syndrome is typified by a very early age at onset (median, 0.5 years) and life-long persistence of periodic fever. Characteristically, attacks occur every 4-8 weeks and continue for 3-7 days, but the individual variation is large. Attacks feature high spiking fever, preceded by chills in 76% of patients. Lymphadenopathy is commonly present (94% of patients). During attacks, 72% of patients complain of abdominal pains, 56% of vomiting, 82% of diarrhea, and 52% of headache. Joint involvement is common in the hyper-IgD syndrome with polyarthralgia in 80% and a non-destructive arthritis, mainly of the large joints (knee and ankle), in 68% of patients.

11. Hyper-IgE Syndrome

Hyper-IgE syndrome (HIES) is a primary immunodeficiency disease characterized by recurrent infections and marked immunoglobulin IgE elevation.

B. Disease of Excess or Aberrant Cytokine Production

Primarily, diseases resulting from the production of excess cytokines are those relating to inflammation (i.e., septic shock, for example, IL-1) or lymphoid and myeloid cancers (e.g., IL-6).

C. Combined Therapy

In another embodiment, it is envisioned that one will treat a B cell immune disease using IL-21 or IL-10+TLR agonist in combination with another therapy, such as an anti-inflammatory or immunosuppressive therapy. Examples of anti-inflammatory compounds include selective (Vioxx™, Celebrex™ and Bextra™) and non-selective NSAIDS. Examples of immunosuppressives include cyclosporin A, FK506, azathioprine, and adrenal corticosteroid hormones.

Combinations may be achieved by contacting cells with a single composition or pharmacological formulation that includes all the agents, or by contacting the cell with multiple distinct compositions or formulations at the same time. Alternatively, the immune therapy of the present invention may precede or follow administration of the anti-inflammatory/immunosuppressive agent by intervals ranging from minutes to weeks. In embodiments where the agents are applied separately to the cell, one would generally ensure that a significant period of time did not expire between the time of each delivery, such that the agents would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an inhibitor or the other agent will be desired. In this regard, various combinations may be employed. By way of illustration, where the IL-21/TLR agonist is “A” and the other therapy is “B,” the following permutations based on 3 and 4 total administrations are exemplary:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are likewise contemplated.

### VIII. B CELL CANCERS

A. B1 B Cell Cancers

1. Chronic Lymphocytic Leukemia

Chronic lymphocytic leukemia (CLL) is a disorder of morphologically mature but immunologically less mature lymphocytes and is manifested by progressive accumulation of these cells in the blood, bone marrow, and lymphatic tissues. Lymphocyte counts in the blood are usually greater than or equal to 5,000/mm³ with a characteristic immunophenotype (CD5⁺ and CD23⁺B cells). For patients with progressing CLL, treatment with conventional doses of chemotherapy is not curative; selected patients treated with allogeneic stem cell transplantation have achieved prolonged disease-free survival. Anti-leukemic therapy is frequently unnecessary in uncomplicated early disease.

CLL occurs primarily in middle-aged and elderly individuals, with increasing frequency in successive decades of life. The clinical course of this disease progresses from an indolent lymphocytosis without other evident disease to one of generalized lymphatic enlargement with concomitant pancytopenia. Complications of pancytopenia, including hemorrhage and infection, represent a major cause of death in these patients. Immunological aberrations, including Coombs-positive hemolytic anemia, immune thrombocytopenia, and depressed immunoglobulin levels may all complicate the management of CLL. Prognostic factors that may help predict clinical outcome include cytogenetic subgroup, immunoglobulin mutational status, ZAP-70, and CD38. Patients who develop an aggressive high-grade non-Hodgkin's lymphoma, usually diffuse large B cell lymphoma and termed a Richter's transformation, have a poor prognosis.

CLL lymphocytes coexpress the B cell antigens CD19 and CD20 along with the T cell antigen CD5. This coexpression only occurs in one other disease entity—mantle cell lymphoma. CLL B cells express relatively low levels of surface-membrane immunoglobulin (compared with normal peripheral blood B cells) and a single light chain (kappa or lambda). CLL is diagnosed by an absolute increase in lymphocytosis and/or bone marrow infiltration coupled with the characteristic features of morphology and immunophenotype, which confirm the characteristic clonal population.

2. Mantle Cell Lymphoma

Mantle cell lymphoma (MCL) is an uncommon type of cancer that makes up only about 5% of all non-Hodgkin's lymphomas. It can occur at any time from the late 30's on, and is three times more common in men than women. The cause of MCL is unknown. The first sign of the disease is often a swelling in the neck, armpit or groin, resulting from enlarged lymph nodes. The cancer may spead to the bone marrow, liver, stomach, colon or spleen.

The most common form of treatment for MCL is chemotherapy. Usually, an intensive chemo regimen is used, with a combination of different drugs being used. Hig dose chemotherapy may be augmented with bone marrow or stem cell infusion. Radiotherapy also may be used when the lymphoma cells are contained in one or two areas of lymph nodes in the same part of the body. Steroids, monoclonal antibodies (rituximab) and interferon also can be used.

B. Combination Therapies

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that IL-21/TLR agonist therapy could be used similarly in conjunction with chemo- or radiotherapeutic intervention. It also may prove effective to combine gene therapy with immunotherapy.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with a IL-21, TLR agonist and at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes all agents, or by contacting the cell with two distinct compositions or formulations, at the same time, wherein one composition includes the IL-21/TLR agonist and the other includes the agent.

Alternatively, the IL-21/TLR agonist treatment may precede or follow the other agent treatment by intervals ranging from minutes to weeks. In embodiments where the other agent and IL-21/TLR agonist are applied separately to the cell, one would generally ensure that a significant period of time did not expire between the time of each delivery, such that the agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either IL-21/TLR agonist or the other agent will be desired. Various combinations may be employed, where IL-21/TLR agonist is “A” and the other agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a IL-21/TLR agonist is particularly preferred as this compound.

In treating cancer according to the invention, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with as described above.

Agents that directly cross-link nucleic acids, specifically DNA, are envisaged to facilitate DNA damage leading to a synergistic, antineoplastic combination with IL-21/TLR agonist. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

### IX. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

A. Materials and Methods

Patients and cell culture. Peripheral blood from 3 healthy subjects, 9 subjects with CD5-positive B-CLL, 2 subjects with CD5-negative B-CLL as well as cord blood from 3 healthy subjects was aquired. The diagnosis of B-CLL required persistent lymphocytosis (>5000 lymphocytes/μl). B-CLL subjects were not under treatment at the time the samples were obtained. Informed consent was obtained from each subject. Mononuclear cells were isolated, and red blood cells removed by resuspending the cells in 5 ml red cell lysis buffer according to standard procedures.

Reagents. The phosphorothioate-modified CpG-motif-containing oligonucleotide ODN 2006 as well as the phosphorothioate-phosphodiester-modified control oligonucleotide ODN 2243 were provided by the Coley Pharmaceutical Group (Wellesley, Mass.). Endotoxin levels in all ODN were <0.075 EU/ml by limulus amebocyte lysate assay. Specific ODN sequences were as follows: ODN 2006: 5′-TCG TCG TTT TGT CGT TTT GTC GTT-3′ (SEQ ID NO:4), ODN 2243: 5′-GGG GGA GCA TGC TGG GGG GG-3′ (SEQ ID NO:5). ODN were diluted in TE (10 mM Tris-HCl, 1 mM EDTA, pH 8) using pyrogen-free reagents. IL-21 was purchased from BioSource (Camarillo, Calif.), IL-2 was purchased from Peprotech (Rocky Hill, N.J.).

Apoptosis and cell survival assay. Cells were harvested at the indicated time points by staining all cells from a given well with mAb for CD19, CD5 and FITC-labeled Annexin V (BD Biosciences, San Diego, Calif.). A predetermined number of calibration beads (CaliBRITE™ Beads, BD Biosciences, San Diego, Calif.) were then added to each sample to allow for normalization of cell counts in different samples at different time points.

Bystander cell assay. Cells were harvested from CLL subjects and separated into aliquots. One aliquot was treated with CpG ODN, IL-21 or both, while a second was untreated but labeled with a membrane dye. Both aliquots were washed thoroughly, and mixed together in the indicated ratio. Cells were harvested after two days, and the number of viable cells in each aliquot determined by gating on the stained or unstained cells.

Gene Profiling—Human Subject Samples. After providing written informed consent, PBMC were obtained from subjects with B-CLL who were not receiving treatment. B-CLL cells were selected using magnetic beads (Miltenyi, Auburn, Calif.). Cells were cultured alone or with 5 μg/ml ODN 2006 in RPMI 1640 media supplemented with 10% fetal bovine serum and 50 μM 2-mercaptoethanol, 2 mM glutamine and penicillin/streptomycin for 2 hours.

Gene Profiling—Isolation of RNA. RNA was isolated using Trizol (Invitrogen, Grand Island, N.Y.) followed by the RNeasy Kit (Qiagen, Valencia, Calif.) used according to the manufacturer's specifications. Briefly, 1 ml Trizol was added per 2×10⁷ cells. After thorough mixing, chloroform was added to 2/10 volume and the specimen shaken for 15 seconds, followed by centrifugation at 12,000 g for 15 minutes at 4° C. The supernatant was transferred into a polypropylene tube, and the volume noted. Ethanol was added slowly to the supernatant during mixing to a final ethanol concentration of 35%. The supernatant was centrifuged at room temperature in an RNeasy midi column, the flow-through collected, and centrifuged again. This was followed by a series of washes with buffers supplied in the kit. DNA was removed as described by the manufacturer (Qiagen, Valencia, Calif.). RNA was eluted from the filter with DEPC treated water and centrifugation. The eluate was aliquoted into 1.5 ml microfuge tubes to which sodium acetate was added (10% volume, 3M, pH 5.2) followed by 2.5 volumes ice-cold ethanol. After at least 15 minutes (but generally overnight at −20° C.), the tubes were centrifuged at 12,000 g at 4° C. for 15 minutes. The pellet was washed twice in 75% ethanol, and resuspended in DEPC-water (20 μl generally, not necessarily 1 mg/ml). The sample was concentrated to 7 mg/ml by centrifugation on a filtration concentrator to clean-up the RNA. The RNA concentration was determined by spectrophotometry and stored at −80° C.

Gene Profiling—Microarray Profiling. Gene expression profiling was conducted by the University of Iowa DNA Core. Gene profiles were generated according to manufacturer guidelines for the U133A chip (Affymetrix, Inc., Santa Clara, Calif.). Quality control test arrays include an Affymetrix test chip containing housekeeping genes (e.g., GAPDH, β-actin) with targets corresponding to various regions of the gene from 3′ to 5′. The core will assess the signal for discrepancies from 3′ to 5′, and if the signal is more than 3-fold different, the sample fails quality control and is prepared again. Intensity values were normalized using Affymetrix MAS 5. After normalization, data analysis was carried out in R (R Development Core Team, 2005). R: A language and environment for statistical computing. R Foundation for Statistical Computing (www.R-project.org).

General statistical analysis. Data are expressed as means±SEM. To determine statistical differences between the means of two data columns, the paired Student's t-test was used. A p value of <0.05 was considered to be significant, a p value of <0.005 was considered to be highly significant. Isobolographic analysis was performed using FlashCalc version 20.5 (FlashCalc Pharmacologic calculations, M. H. Ossipov, Tucson, Ariz.).

B. Results

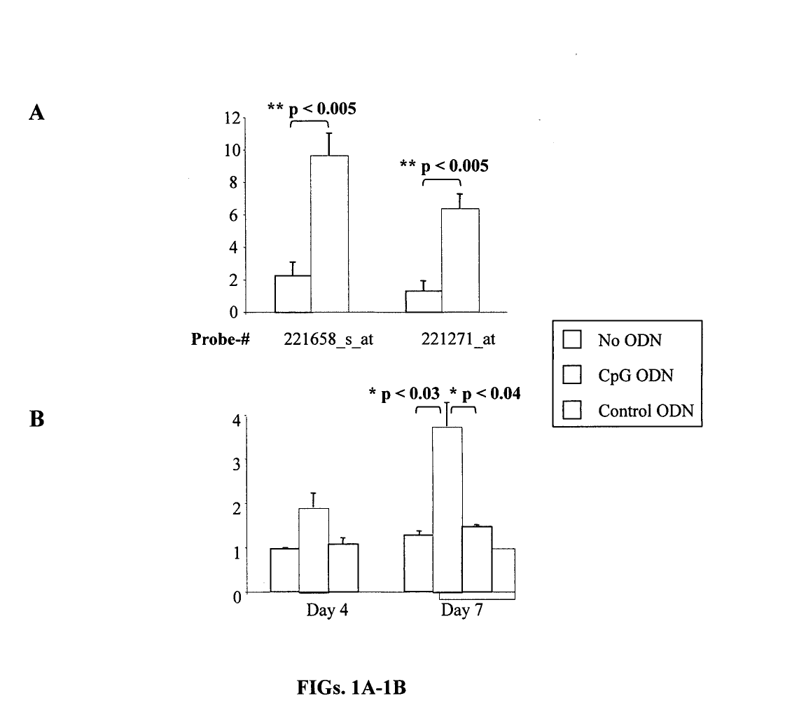

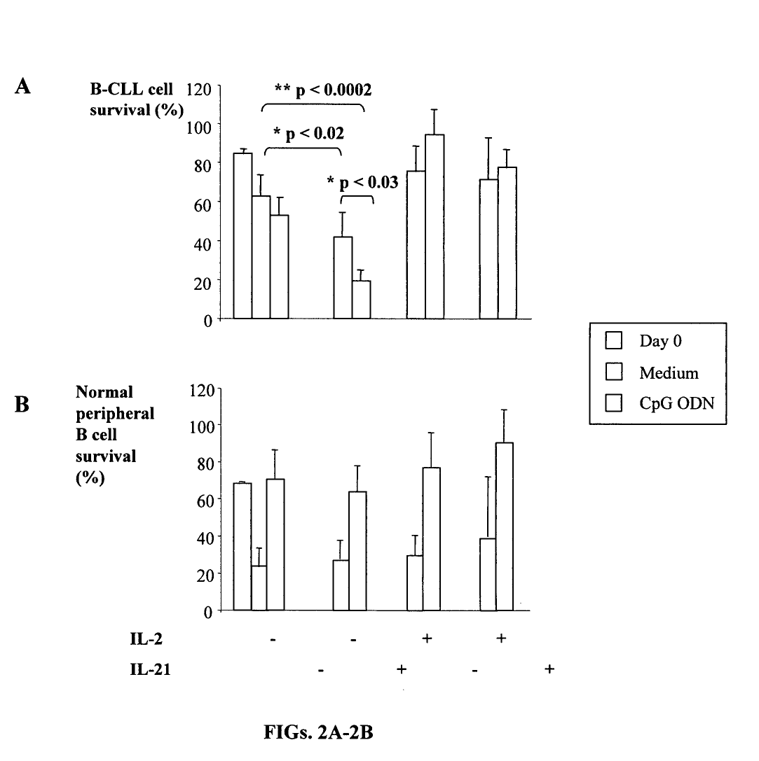

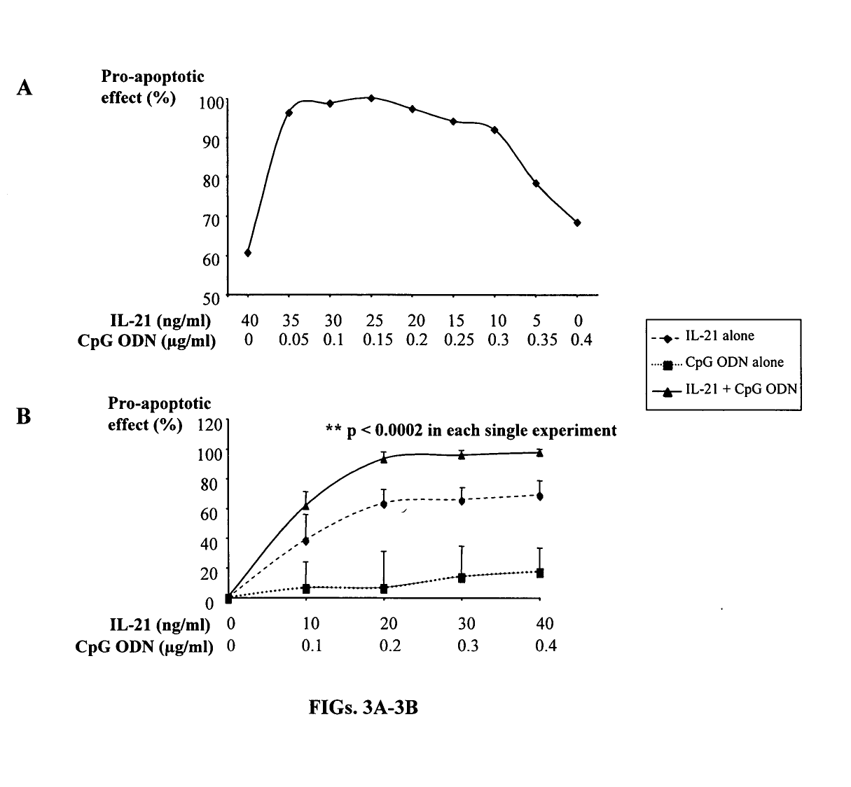

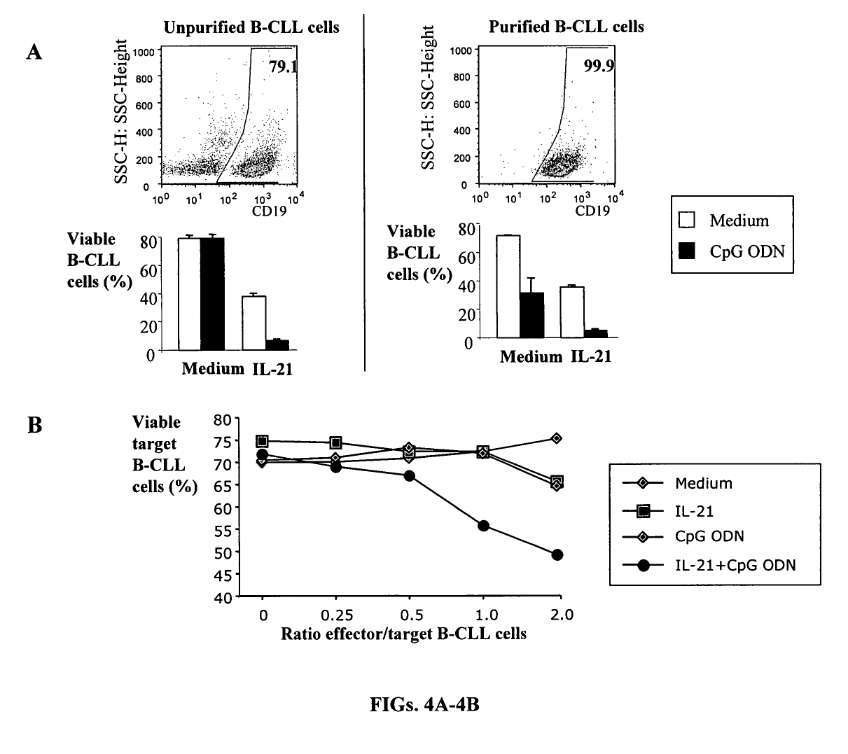

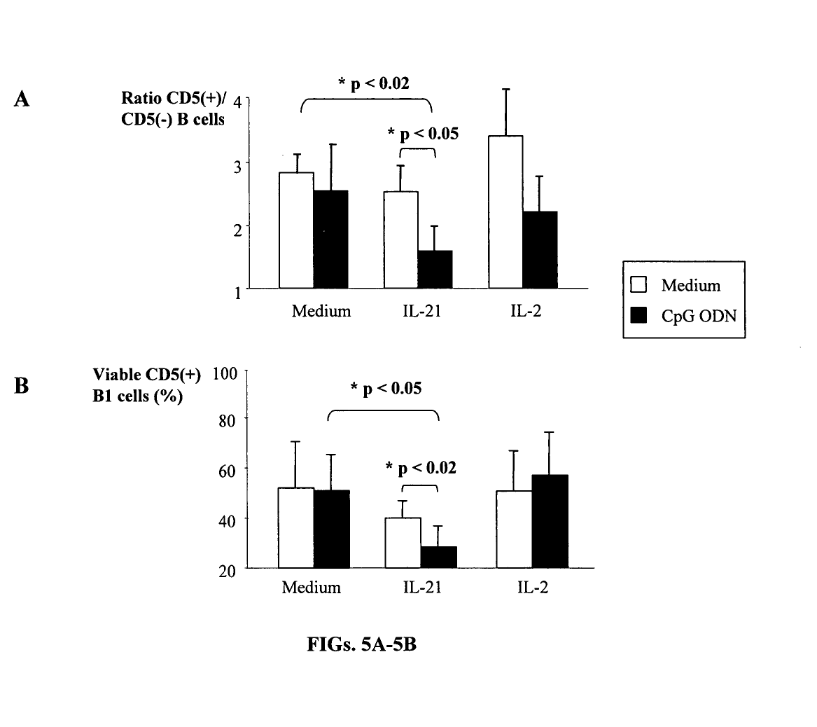

As shown in FIGS. 1A and 1B, CpG ODN increases IL-21 receptor expression by CLL cells, both at the mRNA and protein levels. Incubation with IL-21 and CpG ODN enhances survival of normal B cells, but decreases survival of CLL cells in vitro (FIGS. 2A-2B). CpG ODN and IL-21 are, in fact, synergistic in their ability to induce apoptosis of CLL cells (FIGS. 3-3B). Moreover, CpG ODN and IL-21 induce apoptosis of purified CLL cells (FIGS. 4A-4B), as well as benign CD5(+) B cells from umbilical cord blood (FIGS. 5A-5B).

### Example 3

A. Materials and Methods

Human subjects and cell culture. Peripheral blood from a total of 17 different subjects with B-CLL and 16 different healthy subjects was acquired after obtaining informed consent from each individual. B-CLL subjects were not under treatment at the time the samples were obtained. Mononuclear cells were immediately isolated, and red blood cells removed by resuspending the cells in 5 ml red cell lysis buffer according to standard procedures. In some experiments, CD19-positive B-CLL cells or benign B cells were magnetically purified using the B cell isolation kit I for B-CLL cells and the B cell isolation kit II for benign B cells according to the manufacturer's instructions (Miltenyi Biotec, Auburn, Calif.). During in vitro culture, peripheral blood cells were suspended in AIM-V medium (Gibco BRL, Grand Island, N.Y.) without supplements. Cells were incubated on 96-well-plates (1×10⁶ cells/ml, 200 μl/well, if not stated otherwise) in the presence of different reagents as indicated.

Reagents for functional assays. The phosphorothioate-modified CpG-motif-containing oligodeoxynucleotide ODN 2006 (henceforth referred to as CpG ODN) and the phosphorothioate-phosphodiester-modified control oligodeoxynucleotide 2243 (henceforth referred to as control ODN) were purchased from Coley Pharmaceutical Group (Wellesley, Mass.). Endotoxin levels in all ODN were <0.075 EU/ml by limulus amebocyte lysate assay. Specific ODN sequences were as follows: CpG ODN: 5′-TCG TCG TTT TGT CGT TTT GTC GTT-3′ (SEQ ID NO:4), CONTROL ODN: 5′-GGG GGA GCA TGC TGG GGG GG-3 (SEQ ID NO:5)′. ODN were diluted in TE (10 mM Tris-HCl, 1 mM EDTA, pH 8) using pyrogen-free reagents, and used at a final concentration (fc) of 2.5 μg/ml. Human IL-21 (fc: 100 ng/ml) was purchased from BioSource (Camarillo, Calif.), IL-2 (fc: 100 U/ml) was purchased from Peprotech (Rocky Hill, N.J.). B cell receptor stimulation was performed using affinity purified rabbit F(ab′)₂ fragments against human IgA+IgG+IgM (H+L) (Jackson ImmunoResearch Laboratories, Inc., West Grove, Pa.; fc: 10 μg/ml). For CD40 stimulation mouse mAB against human CD40 (clone B-B20) was used (Diaclone Research, Tepnel Lifecode Corp., Stamford, Conn.; fc: 10 μg/ml). For Granzyme B inhibition experiments carrier- and preservative-free rabbit anti-human Granzyme B polyclonal antibody (IgG) from USBiological (Swampscott, Mass.) was used at the concentrations indicated. ImmunoPure Rabbit IgG from Pierce (Rockford, Ill.) served as control IgG.

Flow cytometry. Antibodies to CD5, CD19, CD27 as well as CD107a were purchased from BD Bioscoences (San Diego, Calif.). IL-21 receptor protein expression was detected using a mAB (clone 152512) from R&D Systems (Minneapolis, Minn.). PKH26 staining was used to identify untreated cells in bystander assays. B-CLL cells were resuspended at a density of 1×10⁷ cells/ml in diluent C containing 2 μM PKH26 (both from Sigma, Saint Louis, Mo.). After 3 minutes, the reaction was stopped by adding an equal volume of FBS (HyClone, Ogden, Utah). Subsequently cells were washed 3 times and finally resuspended in AIM-V medium. For flow cytometric Granzyme and Perforin detection, cells were incubated at 1×10⁶/ml for 13 hrs, Brefeldin A (Epicentre Technologies, Madison, Wis.) added to a final concentration of 1 mg/ml, and cells cultured for 5 more hours. Intracellular staining was performed using a Fix and Perm kit (Caltag Laboratories, Burlingame, Calif.) according to the manufacturer's instructions. Briefly, cells were washed once and resuspended in Fixation Buffer, incubated for 15 minutes at room temperature and washed with PBS. Cells were then resuspended in Permeabilization Buffer and PE- or FITC-labeled antibodies to Granzyme B (clone GB12; Caltag Laboratories), Perforin (clone dG9; BD Pharmingen), Granzyme A (clone CB9; BD Pharmingen) or suitable control antibodies were added. After another 15 minute incubation at room temperature, cells were washed with PBS. Flow cytometric analyses were performed on a FACScan (Becton Dickinson Immunocytometry Systems, San Jose, Calif.) and data analyzed using the program FlowJo (version 6.4.1, Tree Star Inc., Stanford, Calif.).

Flow cytometric apoptosis assays. Cells were stained with Annexin V (BD Biosciences, San Diego, Calif.), or cell-permeable fluorogenic substrates specific for Granzyme B or Caspase 6 for 1 hour at room temperature according to the manufacturer's instructions (Oncolmmunin, Gaithersburg. Md.). A pre-determined number of calibration beads (CaliBRITE™ Beads, BD Biosciences, San Diego, Calif.) was added to each sample to allow for normalization of cell counts at different time points. Propidium iodide at 1 μg/ml was added just prior to flow cytometric analysis. The count of viable cells rather than the count of apoptotic cells was used for the calculation of cell survival because of concerns some non-viable cells may have undergone lysis and not have been available for counting. Absolute cell survival was expressed as percentage of viable cell counts relative to initial plating counts.

Elispot assays for human Granzyme B and Perforin. Human Granzyme B and human Perforin Elispot kits were purchased from Cell Sciences (Canton, Mass.). PVDF-bottomed 96-well plates were purchased from Millipore (Bedford, Mass.). The assays were performed according to the manufacturer's protocol. Briefly, plates were prepared by adding the capture antibody and blocking with 2% skim milk in PBS, then cells were resuspended in AIM-V medium, plated (100 μl/well), and CpG ODN (2.5 μg/ml), IL-21 (100 ng/ml) or both were added and cultured for 16 hours. Freshly isolated PBMC stimulated with PHA (10 μg/ml) for Granzyme B detection and PMA (1 ng/ml) plus lonomycin (500 ng/ml; all from Sigma, St. Louis, Mo.) for Perforin detection served as positive controls. After culture, the detection antibody was added and plates incubated for 1.5 hours. Streptavidin-alkaline phosphatase was distributed, and plates incubated for 1 hr. Finally BCIP/NBT buffer was added and color was allowed to develop for 10 minutes at room temperature followed by rinsing with distilled water. Plates were dried completely, and spots read on an Immunospot Series 1 Analyzer and counted using Immunospot 3 software, both from C.T.L. Cellular Technology Ltd. (Cleveland, Ohio).

TUNEL assay. B-CLL cells were isolated and purified to a percentage of greater than 99.9% using magnetic cell separation. Cells were then suspended at 2×10⁶/ml and plated on a 24-well cell culture plate at 1 ml/well in the presence of different agents as indicated. After 12 hours cells were harvested, fixed in 1% (w/v) paraformaldehyde, suspended at 2×10⁶/ml in 70% (v/v) ethanol and stored for at least 1 hour at −20° C. Fixed cells were stained using the APO-DIRECT™ Kit from BD Biosciences (San Diego, Calif.) according to the manufacturer's instructions. Briefly, cells were washed to remove the ethanol, incubated at 37° C. for 60 minutes in the presence of reaction buffer, TdT Enzyme and FITC-dUTP, rinsed, suspended in PI/RNAse Staining Buffer, and analyzed within 3 hours of staining by flow cytometry.

Isolation of RNA. B-CLL cells were isolated from other cells by magnetic beads as outlined above, and cultured in media or 2.5 μg/ml CpG ODN for 2 hours. RNA was isolated using Trizol (Invitrogen, Grand Island, N.Y.) followed by the RNeasy Kit (Qiagen, Valencia, Calif.) used according to the manufacturer's specifications. Subsequently, the RNA sample was concentrated to 7 mg/ml by centrifugation on a filtration concentrator to clean-up the RNA. The RNA concentration was determined by spectrophotometry and RNA stored at −80° C.

Microarray Profiling. Gene expression profiling was conducted in the University of Iowa DNA Core. Gene profiles were generated according to manufacturer guidelines for the U133A chip (Affymetrix, Inc., Santa Clara, Calif.). Quality control test arrays include an Affymetrix test chip containing housekeeping genes (e.g., GAPDH, β-actin) with targets corresponding to various regions of the gene from 3′ to 5′. The signal was assessed for discrepancies from 3′ to 5′, and if the signal was more than 3-fold different, the sample failed quality control and was prepared again. Intensity values were normalized using Affymetrix MAS 5. After normalization, data analysis was carried out in R (R Development Core 2005).

General statistical analysis. Data are expressed as means±SEM. To determine statistical differences between the means of two data columns, the paired Student's t-test was used. A p value of <0.05 was considered to be significant, a p value of <0.005 was considered to be highly significant. Isobolographic analysis was performed using FlashCalc version 20.5 (FlashCalc Pharmacologic calculations, M. H. Ossipov, Tucson, Ariz.).

B. Results

CpG ODN induces upregulation of the IL-21 receptor by B-CLL cells. The inventors and others have found that CpG ODN can induce apoptosis, and alter the phenotype of B-CLL cells (Jahrsdörfer et al., 2001; Jahrsdörfer et al., 2002; Jahrsdörfer et al., 2005; Jahrsdörfer et al., 2005; Decker and Peschel, 2001; Decker et al., 2000). B-CLL cells also have receptors for, and respond to a variety of IL-2-related cytokines including IL-2, IL-15 and IL-21 de Totero et al., 2006; Decker et al., 2000; Trentin et al., 1996). The inventors therefore evaluated how CpG ODN impacts on the expression of a number of cytokine receptors by B-CLL cells, using gene array and FACS analysis. Among the observed changes was a 4- to 16-fold increase over baseline of the gene for the IL-21 receptor. They also observed a consistent upregulation of IL-21 receptor protein after 4 and 7 days of incubation of the B-CLL cells with CpG ODN. As discussed above, control ODN had no detectable effect on protein levels of IL-21 receptor (FIGS. 1A-1B).

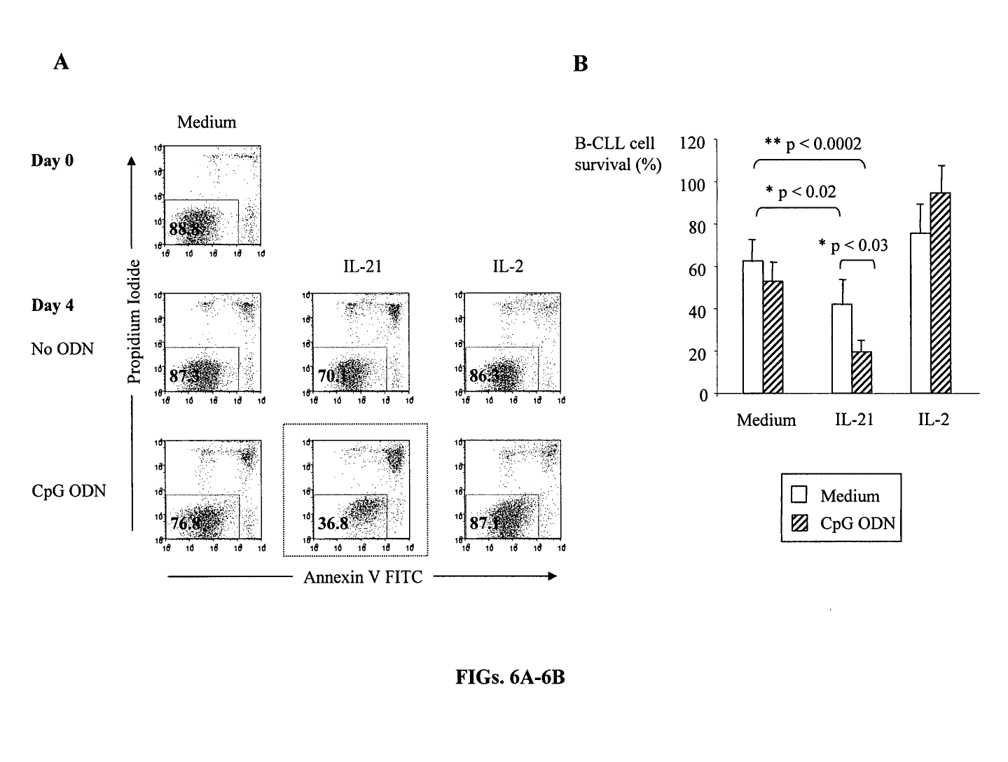

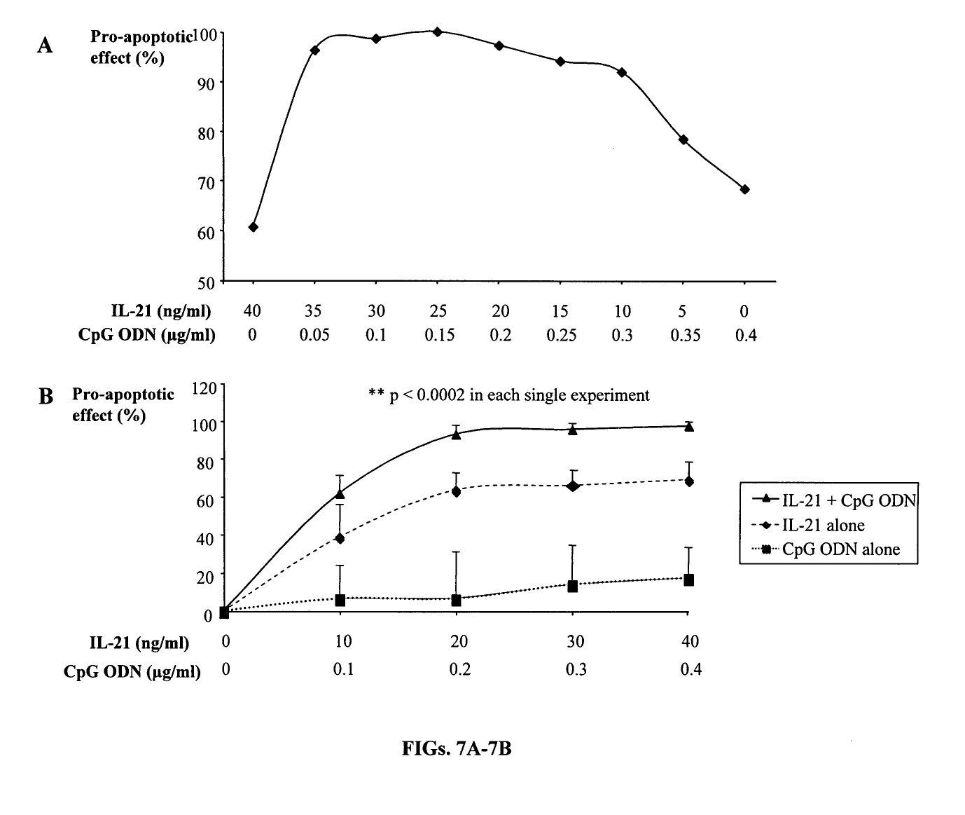

IL-21 and CpG ODN are synergistic in their ability to enhance apoptosis of B-CLL cells. These results prompted us to study the effects on B-CLL cells of IL-21 alone and in combination with CpG ODN. B-CLL cells were isolated and incubated for 4 days in the presence or absence of IL-21 or IL-2. Apoptosis was detected using Annexin V and PI. IL-21 alone induced some apoptosis in B-CLL cells. This effect was strongly enhanced when B-CLL cells were simultaneously treated with CpG ODN (FIGS. 6A-6B). Measurement of apoptosis after 12 hours using DNA fragmentation as measured by flow cytometric TUNEL analysis gave similar results (FIGS. 7A-7B). In contrast to IL-21, IL-2 did not induce apoptosis of B-CLL when combined with CpG ODN. The ability of varying doses of IL-21 and CpG ODN to induce apoptosis was also assessed. The ED₅₀ for each agent alone was identified (IL-21: 40 ng/ml; CpG ODN: 0.4 μg/ml) and the interaction between these agents determined as described by Tallarida et al. (2001). Isobolographic analysis demonstrated that the combined pro-apoptotic effect of IL-21 and CpG ODN on B-CLL cells is synergistic (FIGS. 7A-7B). Engagement of two other B cell stimulating pathways, CD40 or B cell receptor crosslinking, had little effect on the modest pro-apoptotic effect of IL-21 (data not shown).

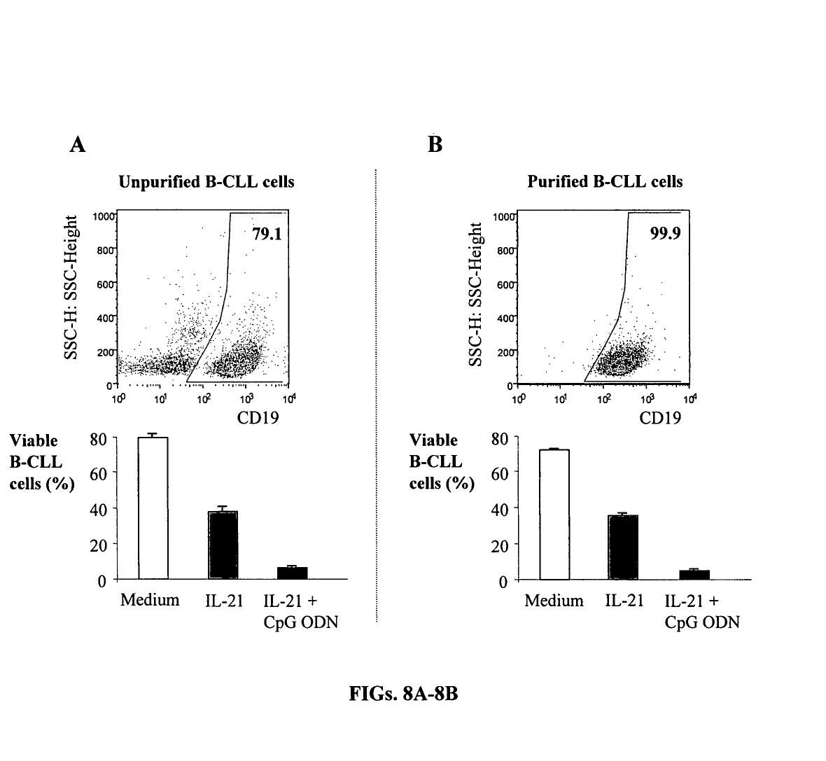

IL-21 and CpG ODN induce apoptosis of highly purified B-CLL cells. One possible explanation for the observed effects on B-CLL cells is that CpG ODN stimulates plasmacytoid dendritic cells (pDCs) or other non-B-CLL cells to produce cytokines such as IFN-α that then impact on the B-CLL cells. To assess this possibility, B cells were purified from B-CLL samples by magnetic bead cell sorting to a purity of >99.9% with less than 0.005% of the remaining cells being pDC. Essentially all sorted cells were CD19(+), CD5(+) suggesting the number of benign B cells in the preparations were very small. As shown in FIGS. 8A-8B, the pro-apoptotic effect of CpG ODN and IL-21 on B cells was similar in unpurified and purified samples suggesting therapy impacts directly on the B-CLL cells, and not secondarily through activation of benign mononuclear cells that are also in blood.

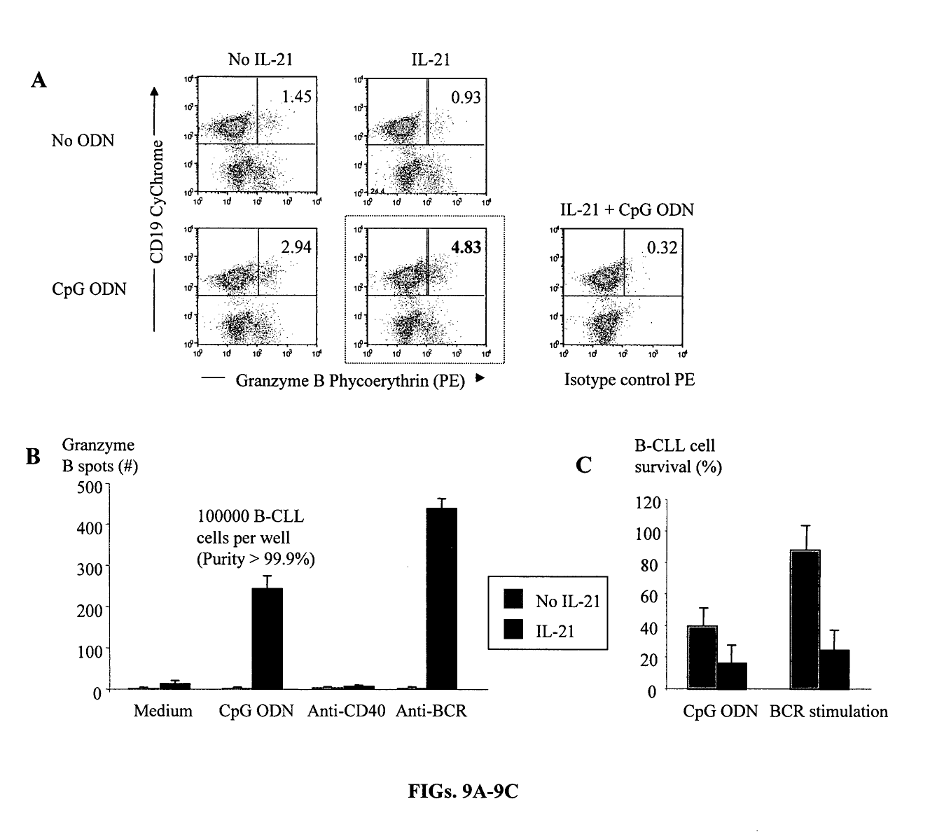

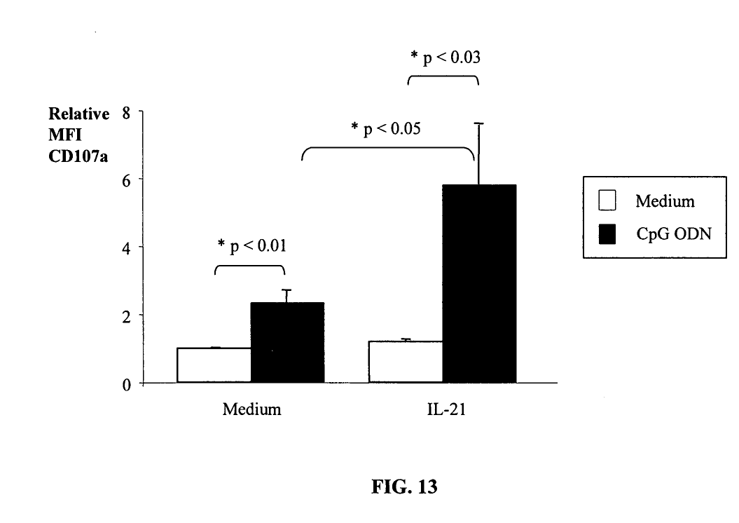

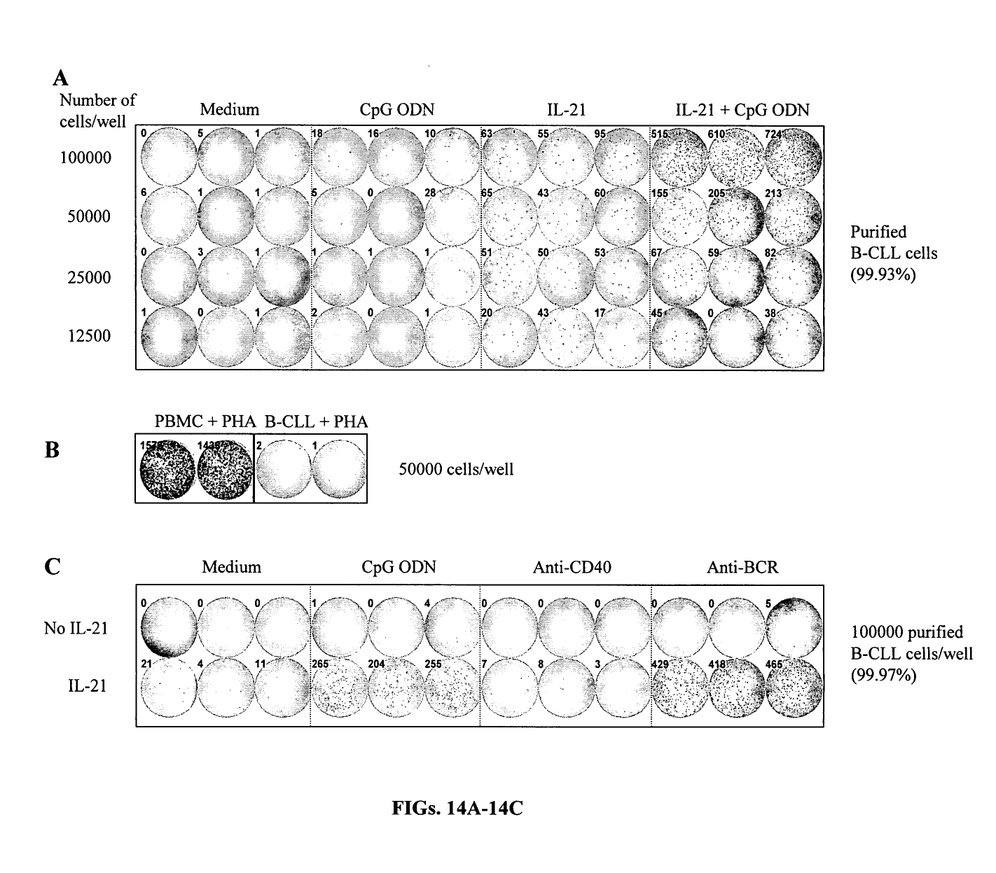

CpG ODN and IL-21 induces expression of CD107a and Granzyme B secretion by B-CLL cells. Further studies were performed to explore the mechanisms behind the observed synergy between IL-21 and CpG ODN. Multicolor flow cytometric analysis demonstrated IL-21 and CpG ODN induced an increase in B-CLL cell granularity (side scatter, data not shown) and enhanced surface expression of lysosomal-associated membrane protein-1 (LAMP-1, CD107a) on B-CLL cells (FIG. 13). Since CD107a is known to be a degranulation marker (Betts et al., 2003; Rubio et al., 2003; Alter et al., 2004), the inventors evaluated the treated B-CLL cells for expression of Granzyme B. Flow cytometric analysis gating on CD19(+) cells revealed rare Granzyme B(+) B cells in samples treated with IL-21 alone. The number of Granzyme B(+) B cells increased significantly in samples treated with the combination of IL-21 and CpG ODN (FIG. 9A). Similar assays demonstrated no detectable Granzyme A or Perforin (data not shown). An ELISpot assay for Granzyme B was used to confirm this finding, and demonstrated that the Granzyme B produced by B-CLL cells was secreted. As with the flow cytometric assay, the ELISpot assay demonstrated IL-21 plus CpG ODN induces Granzyme B production to a greater degree than either agent alone (FIG. 9B, FIG. 14A). As an additional control, samples of purified B cells were treated with PHA, which would be expected to induce Granzyme B secretion by any contaminating T or NK cells. The number of Granzyme B-producing cells in these samples was close to 0, providing further evidence it is the B-CLL cells, and not contaminating T or NK cells, producing the Granzyme B (FIG. 14B).

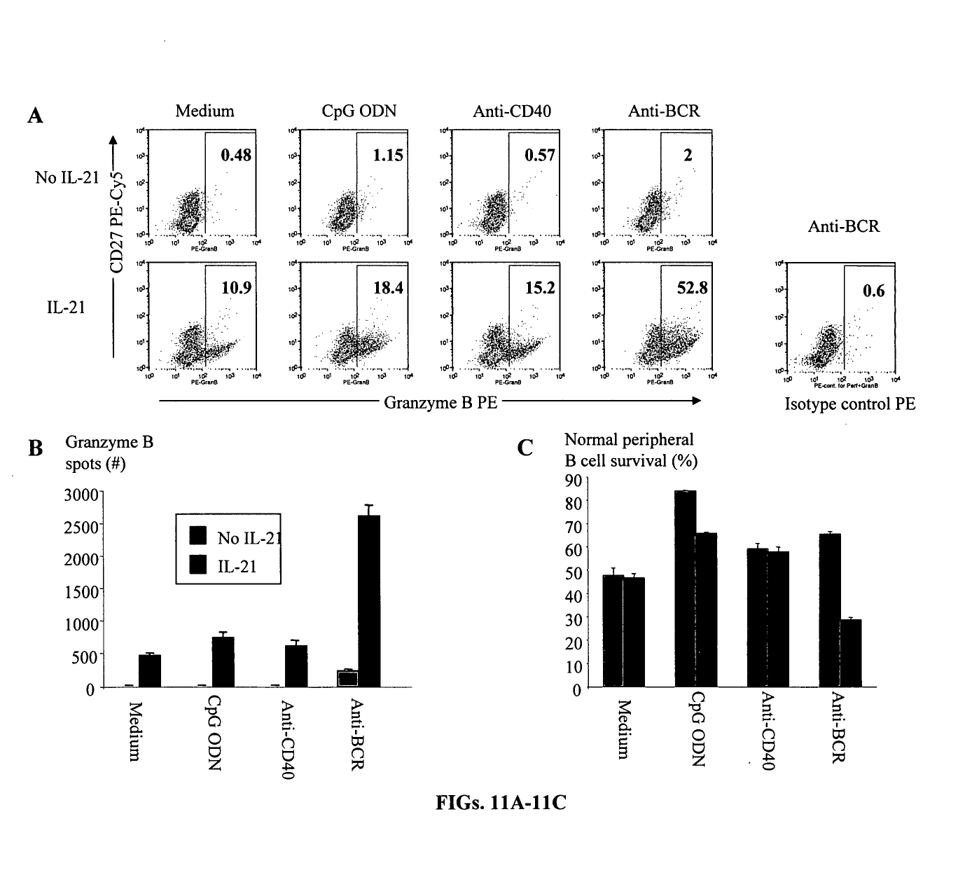

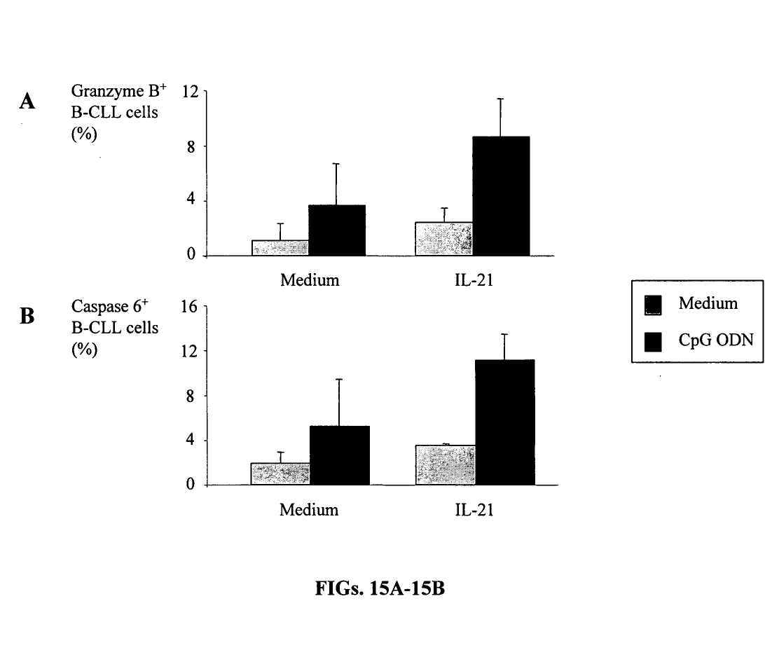

Granzyme B secreted by B-CLL cells is enzymatically active. To assess whether this Granzyme B is functionally active, B-CLL cells were treated with a Granzyme B sensitive cell-permeable substrate. A Caspase 6 sensitive substrate was also evaluated. These substrates fluoresce when cleaved by active Granzyme B or Caspase 6. B-CLL cells were cultured in the presence of IL-21, CpG ODN, or both for 4 days. The above substrates for Granzyme B or Caspase 6 were added for one hour, and samples analyzed by flow cytometry after gating on the CD19(+) cells. As shown in FIGS. 11A-11C, the activity patterns for both, Granzyme B and Caspase 6 were similar to the Granzyme B expression pattern as detected by flow cytometry (FIG. 9A) with the greatest Granzyme B and Caspase 6 activity being seen after treatment of cells with IL-21 and CpG ODN (FIGS. 15A-15B).

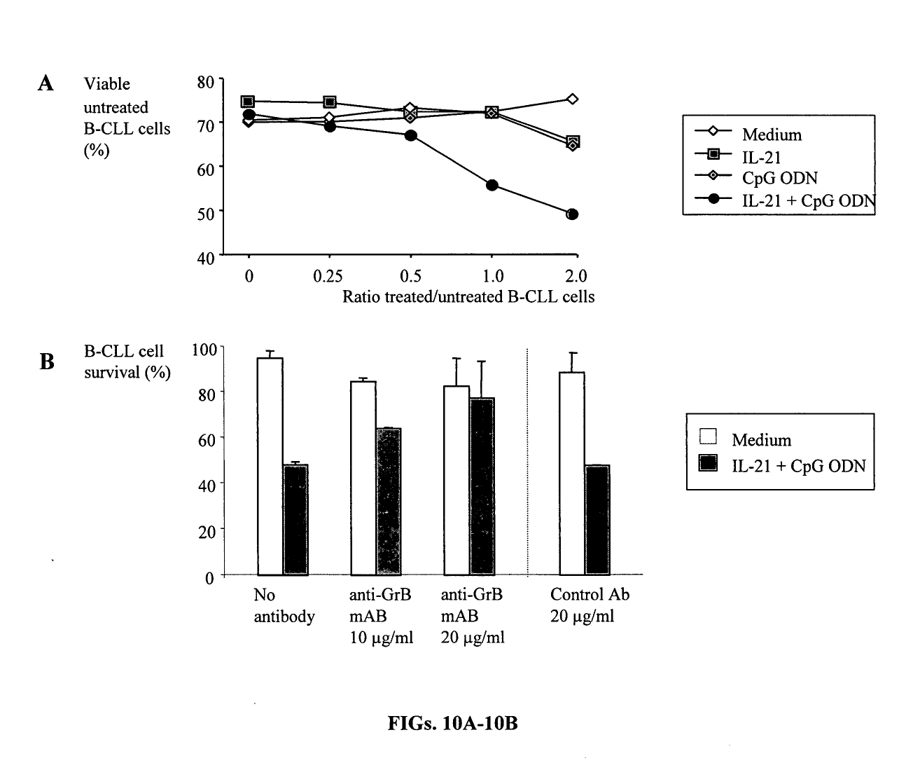

B-CLL cells treated with CpG ODN and IL-21 can kill untreated autologous cells. Bystander killing is blocked by anti-Granzyme B antibody. The inventors next evaluated whether B-CLL cells treated with IL-21 and CpG ODN have the potential to kill. To avoid the complexity of dealing with an allogenic interaction, this was done using autologous B-CLL cells. Purified B-CLL cells were split into two fractions. One fraction was stained with the membrane dye PKH26, and incubated for 24 hours with IL-21 and CpG ODN. These stained, treated cells were washed three times to remove the agents, and mixed with the unstained, untreated fraction for 2 days. The survival of the unstained, untreated cells was then evaluated by flow cytometry. As illustrated in FIG. 10A, the cells treated with IL-21 and CpG ODN induced apoptosis of the untreated cells, indicating B-CLL cells treated with IL-21 and CpG ODN were capable of killing untreated B-CLL cells. The addition of anti-human Granzyme B antibody inhibited this bystander killing in a dose-dependent manner (FIG. 10B), providing further evidence that Granzyme B was involved in the observed cell death.

Effect of IL-21 and other B cell activators on B-CLL cells. Combinations of other B cell stimulatory agents were tested for their ability to induce secretion of Granzyme B by B-CLL cells. A stimulating anti-B cell receptor (BCR) antibody, but not an anti-CD40 antibody, enhanced IL-21-induced Granzyme B secretion by B-CLL cells. (FIG. 9B, FIG. 14C). While BCR stimulation alone protected B-CLL cells from spontaneous apoptosis much stronger than CpG ODN, the IL-21-mediated pro-apoptotic effect was similar in BCR-stimulated as compared to CpG ODN-activated B-CLL cells (FIG. 9C). Substitution of IL-21 by IL-2 did not induce secretion of Granzyme B in B-CLL cells nor did it induce B-CLL cell apoptosis (data not shown and FIG. 6B).

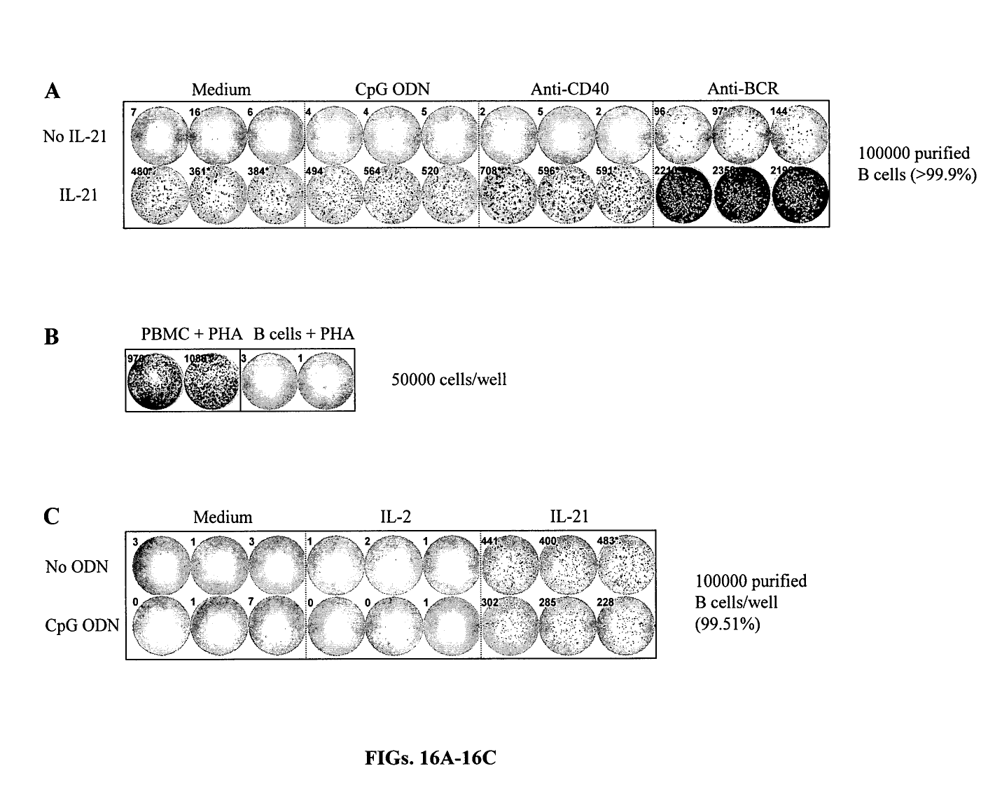

Effect of IL-21 and B cell activators on benign B cells. Benign peripheral blood B cells from normal donors were evaluated for their response to IL-21 and CpG ODN using the assays outlined above. IL-21 induced expression of Granzyme B by highly purified (99.9%) B cells as demonstrated by both flow cytometry and ELISpot assay (FIGS. 11A-11B and FIGS. 16A-16B). Stimulating antibodies to the BCR, but not antibodies to CD40 or CpG ODN, strongly enhanced IL-21-induced Granzyme B secretion by benign B cells. Staining of purified B cells with anti-CD27 revealed it was mainly the CD27(−) naïve B cell population which produced Granzyme B (FIG. 11A). Neither IL-21 alone nor IL-21 plus CpG ODN induced apoptosis of benign B cells, however the combination of IL-21 and BCR crosslinking resulted in a decreased number of viable B cells after 3 days of culture (FIG. 11C). Thus, both activated benign B cells and B-CLL cells produce and secrete Granzyme B in response to IL-21. In contrast, B-CLL cells undergo apoptosis in response to IL-21 plus CpG ODN while benign B cells do not.

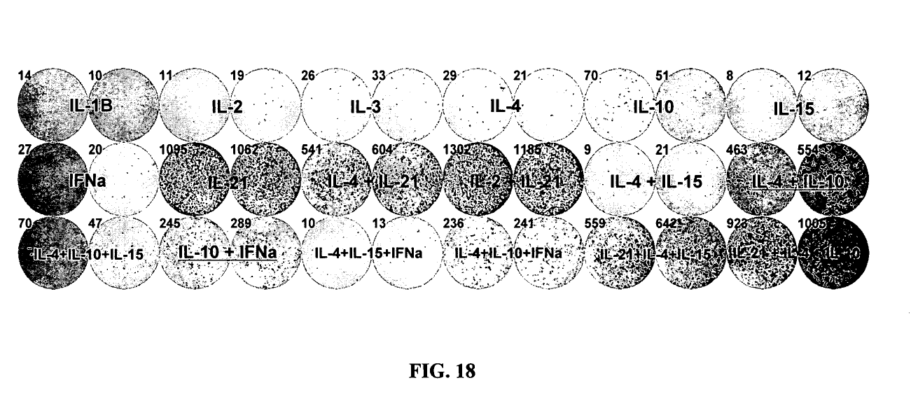

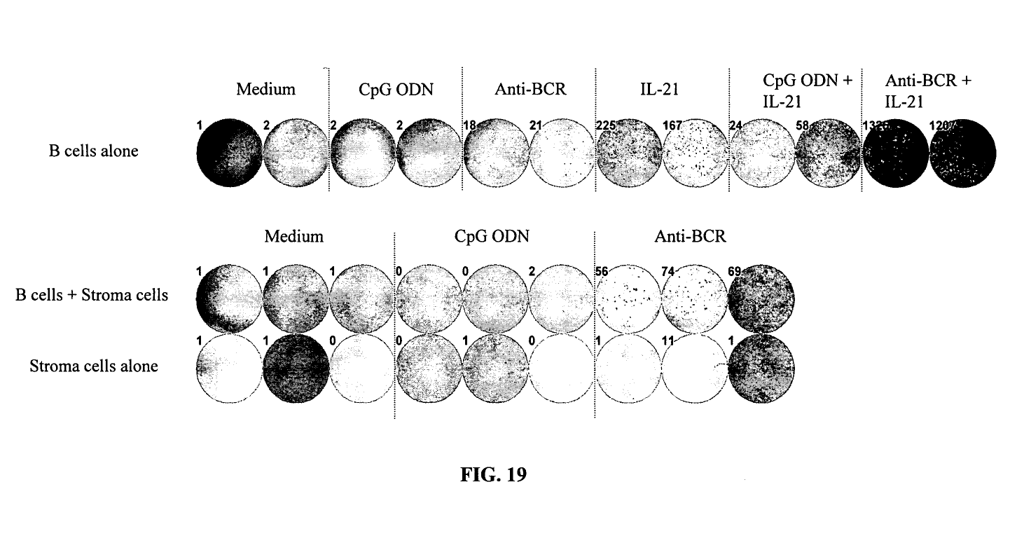

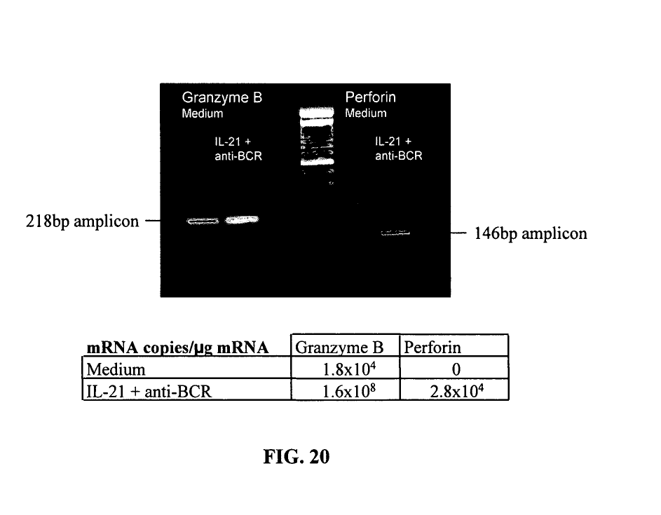

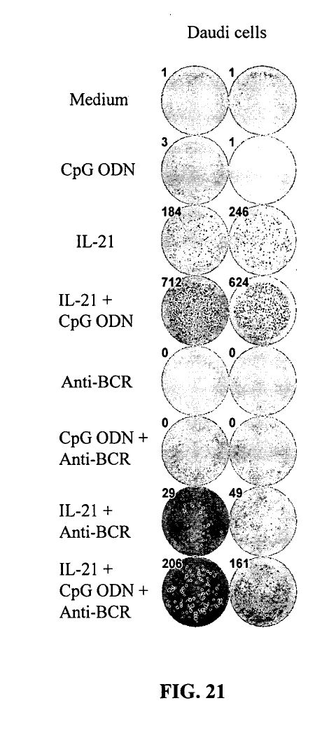

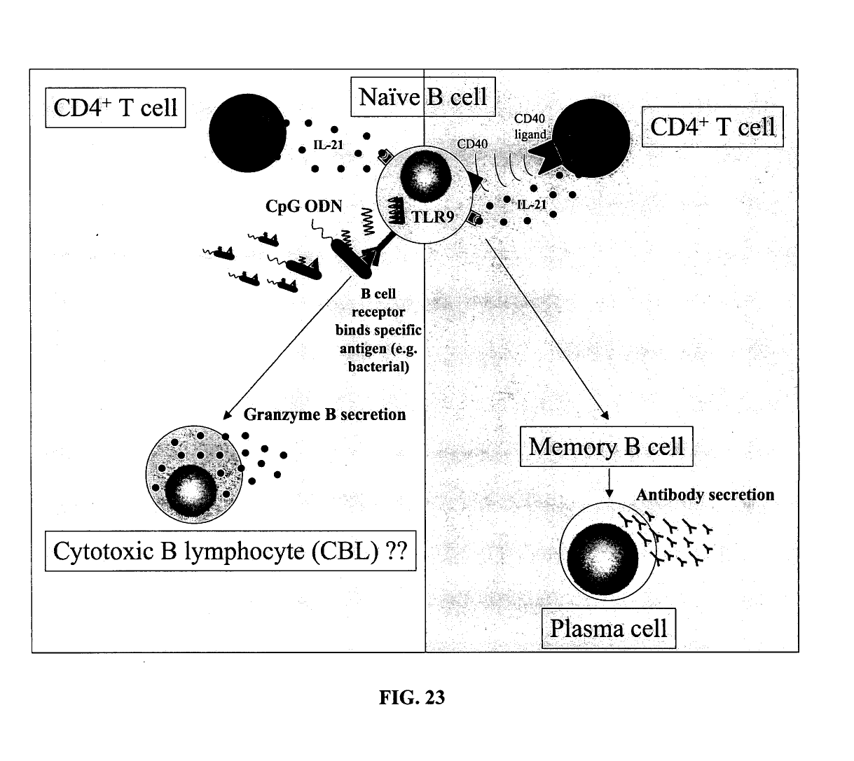

In-vitro data concerning the development of cytotoxic B cells. FIG. 23 illustrates the general overview of the inventors understanding of the development of cytotoxic B cells. New findings are that IL-21 is not the only cytokine that can induce the cytotoxic differentiation pathway in B cells. The inventors have identified at least two cytokine combinations so far (IL-10+IL-4 and IL-10+IFN-alpha, FIG. 18) with similar effects. Another finding is that bone marrow stroma cells can also induce B cells to produce granzyme B (FIG. 19). This is indicative of the physiological function of the inventors findings since it could represent a way how autoreactive B cells may be deleted in the bone marrow. More evidence for a potential cytotoxic function of B cells comes from the new findings that B cells can also produce perforin (FIG. 20) and interferon-gamma (FIG. 21). Last but not least B cells are able to inhibit the expansion of CD4-positive T cells in the presence of IL-21, CpG ODN and B cell receptor stimulation which is indicative of a possible regulatory function of B cells in-vivo.

C. Discussion

IL-21 is an IL-2 family cytokine, mainly produced by activated CD4+T cells, and with pleiotropic effects on T, B and NK cells (Parrish-Novak et al., 2000; Habib et al., 2003; Mehta et al., 2004). One effect of IL-21 is to upregulate the Granzyme A and B genes by cytotoxic CD8+T cells (Leonard and Spolski, 2005; Zeng et al., 2005). Apart from CTL and NK cells, no other human lymphocyte populations are known to produce and secrete Granzymes at biologically active levels. In the studies above, the inventors demonstrated that activated human B cells can produce and secrete Granzyme B in response to IL-21. This effect can be enhanced by B cell receptor (BCR) crosslinking, as well as the TLR 9 agonist CpG ODN.

The combination of IL-21 plus CpG ODN is cytotoxic to B-CLL cells, and the Granzyme B produced by treated B-CLL cells can kill untreated autologous bystander B-CLL cells. This effect is blocked in part by anti-Granzyme B, confirming that at least some of the observed effect results from the cytotoxic effects of secreted Granzyme B. Since efficient cell-permeable small molecule inhibitors of Granzyme B do not yet exist (Russell and Ley, 20020, the inventors cannot exclude that apart from fratricidal also suicidal killing plays a role in the observed pro-apoptotic effect in B-CLL cells, comparable to what has been reported in NK cells, where leakage of cytotoxic granules into the cytoplasm can induce suicidal NK cell apoptosis (Ida et al., 2003). On the other hand, only little enhancement of apoptosis was seen after treatment of benign B cells with anti-BCR plus IL-21 despite the high levels of Granzyme B produced by such cells. Thus, Granzyme B production alone may not be the only determinant of the cytotoxic potential of B cells.

Indeed, many other factors can impact on whether Granzyme B is able to kill or not. First of all, effector and target cells need to get into close contact to each other and form a secretory synapse involving a series of receptors and ligands on both the effector and the target cell side (Bossi et al., 2002). A striking difference between benign B cells and B-CLL cells is the expression of the T cell marker CD5 on B-CLL cells. CD5 is associated with an immunoregulatory tyrosine-based inhibitory motif which generally may explain why B cells that express CD5 can respond differently to various stimuli (Bikah et al., 1996; Pers et al., 2002). Furthermore, CD5 has several ligands including CD72, which is also expressed by B-CLL cells (Garand et al., 1994). It is therefore possible that these molecules enhance the interaction between B-CLL cells, allowing Granzyme B to be transferred more efficiently. Indeed, preliminary data from the inventors' laboratory suggests blocking CD5 can partially inhibit IL-21-induced apoptosis in CD5-positive CpG-activated B-CLL, while CD5-negative, atypical cases of B-CLL do not undergo apoptosis in response to IL-21 (unpublished results).

Second, killing mediated by Granzyme B is generally associated with perforin which allows release of Granzyme B from endosomes into the cytosol after uptake into the target cell. The inventors found no evidence for production of perforin by treated B cells. Although perforin levels below the detection limit may have been present in this case, Granzyme B-mediated apoptosis is principally possible in the absence of perforin and the presence of various microbial products (Froelich et al., 1996; Browne et al., 1999; Kurschus et al., 2004; Choy et al., 2004). One well-known example is, that co-internalization of Granzyme B with adenovirus, a virus that escapes endosomes to reach the cytosol, allows Perforin-independent delivery of Granzyme B into the cytoplasm and subsequently full induction of apoptosis (Froelich et al., 1996). If an unknown endosomolytic agent (either exogenous or endogenous) would be present in B-CLL cells but not in benign B cell, this could explain the differences in their apoptotic response to IL-21.

Third, another possible approach to explain the different apoptotic response to IL-21 between benign B cells and B-CLL cells could be sensitivity. Cells that produce Granzyme B are known to express proteins, such as proteinase inhibitor 9 (PI-9), that protect the cells from the pro-apoptotic effects of Granzyme B (Sun et al., 1996). However, malignant B cells that express PI-9 can still be sensitive to cytotoxic granule-mediated apoptosis (Godal et al., 2005), suggesting expression of such proteinases is not a guarantee against apoptosis and that benign B cells might have evolved other, so far unknown protective mechanisms against Granzyme B-mediated apoptosis, which are defect in B-CLL cells.

Additional studies are needed before one can fully understand which factors determine whether B cells will produce functional Granzyme B in response to IL-21, and the impact of IL-21-induced B cell Granzyme B on immunity. A number of possibilities need to be considered. First, B cells activated by IL-21 and other B cell stimuli could undergo apoptosis after producing Granzyme B, thus providing a negative feedback loop that limits excessive B cell activation. This could be particularly interesting for the understanding of B cell selection processes in the bone marrow. Second, negative feedback could also result if Granzyme B produced by B cells induces apoptosis of the CD4+T cell that produced the IL-21. On the other hand, cytotoxic B cells could be part of a positive feed back response to significant infection when multiple activation signals are present in a local environment. More specifically, local bacterial infection could lead to B cell activation by BCR crosslinking or TLR9 activation by microbial DNA, in the same microenvironment where IL-21 is being produced by activated CD4+T cells. B cells stimulated under such conditions would become cytotoxic towards cells expressing target antigen, independent of MHC, thereby resulting in an accelerated cytotoxic response against microbial intruders. In contrast to CpG ODN treatment or BCR ligation, CD40 ligation did not enhance Granzyme B production by IL-21-treated B cells. A recent report suggests IL-21 plus engagement of CD40 can induce B cells to terminally differentiate into plasma cells while IL-21 plus B cell receptor crosslinking does not (Ettinger et al., 2005). This finding, combined with the results reported here, suggests CD40 ligation plus IL-21 could provide the environment for naïve B cell differentiation and a longer term, adaptive and systemic immune response. In contrast, a micro-environment that results in exposure of the naïve B cells to IL-21 plus BCR cross-linking (or CpG ODN) might signal for a more rapid, local, and undirected immune response and development of B cells with cytotoxic potential (FIG. 23).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,166,452
U.S. Pat. No. 4,256,108
U.S. Pat. No. 4,265,874
U.S. Pat. No. 4,659,774
U.S. Pat. No. 4,682,195
U.S. Pat. No. 4,683,202
U.S. Pat. No. 4,816,571
U.S. Pat. No. 4,843,092
U.S. Pat. No. 4,959,463
U.S. Pat. No. 5,141,813
U.S. Pat. No. 5,264,566
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,705,629
U.S. Pat. No. 5,968,909
U.S. Pat. No. 6,218,371
U.S. Pat. No. 6,339,068
U.S. Pat. No. 6,406,705
U.S. Pat. No. 6,498,148
U.S. Pat. No. 6,544,518
U.S. Pat. No. 6,558,670
U.S. Pat. No. 6,562,798
U.S. Pat. No. 6,589,940
U.S. Pat. No. 6,610,661
U.S. Pat. No. 6,667,293
U.S. Pat. No. 6,821,957
Alter et al., J. Immunol. Methods, 294:15-22, 2004.
Angel et al., Cell, 49:729, 1987b.
Angel et al., Mol. Cell. Biol., 7:2256, 1987a.
Arend and Dayer, Arthritis Rheum., 38:151-160, 1995.
Arend et al., Annu. Rev. Immunol., 16:27-55, 1998.
Atchison and Perry, Cell, 46:253, 1986.
Atchison and Perry, Cell, 48:121, 1987.
Baichwal and Sugden, In: Gene Transfer, Kucherlapati (Ed.), NY, Plenum Press, 117-148, 1986.
Banerji et al., Cell, 27(2 Pt 1):299-308, 1981.
Banerji et al., Cell, 33(3):729-740, 1983.
Benvenisty and Neshif, Proc. Natl. Acad. Sci. USA, 83(24):9551-9555, 1986.
Berkhout et al., Cell, 59:273-282, 1989.
Betts et al., J. Immunol. Methods, 281:65-78, 2003.
Bikah et al., Science, 274:1906-1099, 1996.
Blanar et al., EMBO J., 8:1139, 1989.
Bodine and Ley, EMBO J., 6:2997, 1987.
Boshart et al., Cell, 41:521, 1985.
Bossi et al., Immunol. Rev., 189:152-160, 2002.
Bosze et al., EMBO J., 5(7):1615-1623, 1986.
Braddock et al., Cell, 58:269, 1989.
Browne et al., Mol. Cell Biol., 19:8604-8615, 1999.
Brunet et al., Nature, 322:268-271, 1986.
Bulla and Siddiqui, J. Virol., 62:1437, 1986.
Burger and Dayer, Neurology, 45(6S-6):S39-43, 1995.
Campbell and Villarreal, Mol. Cell. Biol., 8:1993, 1988.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Celander and Haseltine, J. Virology, 61:269, 1987.
Celander et al., J. Virology, 62:1314, 1988.
Chandler et al., Cell, 33:489, 1983.
Chang et al., Hepatology, 14:134A, 1991.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7(8):2745-2752, 1987.
Choi et al., Cell, 53:519, 1988.
Choy et al., Arterioscler. Thromb. Vasc. Biol., 24:2245-2250, 2004.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Crosby et al., Genomics, 6:252-259, 1990.
Culotta and Hamer, Mol. Cell. Biol., 9:1376, 1989.
Culver et al., Science, 256(5063):1550-1552, 1992.
Dahl et al., Hum. Genet., 94:465-470, 1990.
Dandolo et al., J, Virology, 47:55-64, 1983.
de Totero et al., Blood, 3:3, 2006.
De Villiers et al., Nature, 312(5991):242-246, 1984.
Decker and Peschel, Leuk. Lymphoma., 42:301-307, 2001.
Decker et al., Blood, 95:999-1006, 2000.
Decker et al., Exp. Hematol., 28:558-568, 2000.
Deschamps et al., Science, 230:1174-1177, 1985.
Dinarello, Int. Rev. Immunol., 16:457-499, 1998.
Dubensky et al., Proc. Natl. Acad. Sci. USA, 81:7529-7533, 1984.
Eastgate et al., Lancet, 2:706-709, 1988.
Edbrooke et al., Mol. Cell. Biol., 9:1908, 1989.
Edlund et al., Science, 230:912-916, 1985.
EPO 0273085
EPO 266 032
Ettinger et al., J. Immunol., 175:7867-7879, 2005.
Fechheimer, et al., Proc Natl. Acad. Sci. USA, 84:8463-8467, 1987.
Feng and Holland, Nature, 334:6178, 1988.
Ferkol et al., J. Clin. Invest., 92:2394-2400, 1993.
Firak and Subramanian, Mol. Cell. Biol., 6:3667, 1986.
Firestein et al., Arthritis Rheum., 37:644-652, 1994.
Foecking and Hofstetter, Gene, 45(1):101-105, 1986.
Fraley et al., Proc. Natl. Acad. Sci. USA, 76:3348-3352, 1979.
Friedmann, Science, 244:1275-1281, 1989.
Froehler et al., Nucleic Acids Res., 14(13):5399-5407, 1986.
Froelich et al., J. Biol. Chem., 271:29073-29079, 1996.
Fujikawa et al., Ann. Rheum. Dis., 54:318-320, 1995.
Fujita et al., Cell, 49:357, 1987.
Garand et al., Leuk. Res., 18:651-652, 1994.
Ghosh and Bachhawat, In: Liver Diseases, Targeted Diagnosis and Therapy Using Specific Receptors and Ligands, Wu et al. (Eds.), Marcel Dekker, N.Y., 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godal et al., Blood, 22:22, 2005.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Goodman and Gilman's The Pharmacological Basis Of Therapeutics, Hardman et al. (Eds.), 10^{th} Ed., 32:853-860; 35:891-893, 2001.
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. Gen. Virl., 36(1):59-74, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Habib et al., J. Allergy Clin. Immunol., 112:1033-1045, 2003.
Haddad et al., Gene, 87:265-271, 1990.
Hahn and Tsao, In: Dubois' Lupus Erythematosus, 4^{th} Ed, Wallace and Hahn (Eds.), Lea and Febiger, Philadelphia, 195-201, 1993.
Hannum et al., Nature, 343:336-340, 1990.
Hanson et al., Proc. Natl. Acad. Sci. USA, 87:960-963, 1990.
Harland and Weintraub, J. Cell Biol., 101(3):1094-1099, 1985.
Harper et al., Immunogenetics, 28:439-444, 1988.
Haslinger and Karin, Proc. Natl. Acad. Sci. USA, 82:8572, 1985.
Hauber and Cullen, J, Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., 1990
Herz and Gerard, Proc. Natl. Acad. Sci. USA, 90:2812-2816, 1993.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Horwich et al. J. Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Ida et al., Eur. J. Immunol., 33:3284-3292, 2003.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jahrsdorfe et al., J. Leukoc. Biol., 72:83-92, 2002.
Jahrsdorfer et al., Clin. Cancer Res., 11: 1490-1499, 2005.
Jahrsdorfer et al., J. Leukoc. Biol., 69:81-88, 2001.
Jahrsdorfer et al., J. Leukoc. Biol., 77:378-387, 2005.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kahle et al., Ann. Rheum. Dis., 51:731-734, 1992.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al, J. Biol. Chem., 266:3361-3364, 1991.
Kawamoto et al., Mol. Cell. Biol., 8:267, 1988.
Kiledjian et al., Mol. Cell. Biol., 8:145, 1988.
Klamut et al., Mol. Cell. Biol., 10:193, 1990.
Klein et al., Genomics, 5:110-117, 1989.
Klein et al., Nature, 327:70-73, 1987.
Koch et al., Mol. Cell. Biol., 9:303, 1989.
Kotzin and O'Dell, In: Samler's Immunologic Diseases, 5^{th} Ed., Frank et al. (Eds.), Little Brown & Co., Boston, 667-697, 1995.
Kotzin, Cell, 85:303-306, 1996.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Kurschus et al., FEBS Lett., 562:87-92, 2004.
Larsen et al., Proc. Natl. Acad. Sci. USA, 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Lee et al., Nucleic Acids Res., 12:4191-206, 1984.
Leonard and Spolski, Nat. Rev. Immunol., 5:688-698, 2005.
Levinson et al., Nature, 295:79, 1982.
Levrero et al., Gene, 101:195-202, 1991.
Lin et al., Cytogenet. Cell Genet., 53:169-171, 1990.
Lin et al., Mol. Cell. Biol., 10:850, 1990.
Lipsky, In: Harrison's principles of internal medicine, Fauci et al.(Eds.), 14^{th} Ed., NY, McGraw-Hill, 1880-1888, 1998.
Lord et al., Immunol. Rev., 193:31-38, 2003.
Luria et al., EMBO J., 6:3307, 1987.
Lusky and Botchan, Proc. Natl. Acad. Sci. USA, 83:3609, 1986.
Lusky etal., Mol. Cell. Biol., 3:1108, 1983.
Maas et al., J. Immunol., 162:6526-6553, 1999.
Macejak and Sarnow, Nature, 353:90-94, 1991.
Majors and Varmus, Proc. Natl. Acad. Sci. USA, 80:5866, 1983.
Mann et al., Cell, 33:153-159, 1983.
Markowitz et al., J. Virol., 62:1120-1124, 1988.
McNeall et al., Gene, 76:81, 1989.
Mehta et al., Immunol. Rev., 202:84-95, 2004.
Miksicek et al., Cell, 46:203, 1986.
Mordacq and Linzer, Genes and Dev., 3:760, 1989.
Moreau et al., Nucl. Acids Res., 9:6047, 1981.
Muesing et al., Cell, 48:691, 1987.
Ng et al., Nuc. Acids Res., 17:601, 1989.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Ohnishi et al., Int. Immunol., 6:817-830, 1994.
Ondek et al., EMBO J., 6:1017, 1987.
Omitz et al., Mol. Cell. Biol., 7:3466, 1987.
Palmiter et al., Nature, 300:611, 1982.
Parrish-Novak et al., Nature, 408:57-63, 2000.
Paskind et al., Virology, 67:242-248, 1975.
Pech et al., Mol. Cell. Biol., 9:396, 1989.
Pelletier and Sonenberg, Nature, 334(6180):320-325, 1988.
Perales et al., Proc. Natl. Acad. Sci. USA, 91:4086-4090, 1994.
Perez-Stable and Constantini, Mol. Cell. Biol., 10:1116, 1990.
Pers et al., Leukemia, 16:44-52, 2002.
Physicians Desk Reference
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10: 1076, 1990.
Potter et al., Proc. Natl. Acad. Sci. USA, 81:7161-7165, 1984.
Prieur et al., Lancet., 2:1240-1242, 1987.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham:

Butterworth, pp. 467-492, 1988.
Ripe et al., Mol. Cell. Biol., 9:2224, 1989.
Rippe, et al., Mol. Cell Biol., 10:689-695, 1990.
Rissoan et al., Blood, 100:3295-3303, 2002.
Rittling et al., Nuc. Acids Res., 17:1619, 1989.
Robertson et al., Nature 322:445-448, 1986
Rooney et al., Rheumatol. Int., 10:217-219, 1990.
Rosenetal., Cell, 41:813, 1988.
Rosenfeld et al., Science, 252:431-434, 1991.
Rosenfeld, et al., Cell, 68:143-155, 1992.
Roux et al., Proc. Natl. Acad. Sci. USA, 86:9079-9083, 1989.
Rubio et al., Nat. Med., 9:1377-1382, 2003.
Russell and Ley, Annu. Rev. Immunol., 20:323-370, 2002.
Sakai et al., Genes and Dev., 2:1144, 1988.
Sambrook et al., In: Molecular cloning, Cold Spring Harbor Laboratory Press, Cold Spring Harbor, N.Y., 2001.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Sun et al., J. Biol. Chem., 271:27802-27809, 1996.
Swartzendruber and Lehman, J. Cell. Physiology, 85:179, 1975.
Takebe et al., Mol. Cell. Biol., 8:466, 1988.
Tallarida, J. Pharmacol. Exp. Ther., 298:865-872, 2001.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
The Merck Index, O'Neil et al., ed., 13^{th} Ed., 2001.
Thiesenetal., J. Virology, 62:614, 1988.
Top et al., J. Infect. Dis., 124:155-160, 1971.
Trapani and Sutton, Curr. Opin. Immunol., 15:533-543, 2003.
Treisman, Cell, 42:889, 1985.
Trentin et al., Blood, 87:3327-3335, 1996.
Tronche et al., Mol. Biol. Med., 7:173, 1990.
Trudel and Constantini, Genes and Dev., 6:954, 1987.
Tur-Kaspa et al., Mol. Cell Biol., 6:716-718, 1986.
Tyndell et al., Nuc. Acids. Res., 9:6231, 1981.
van den Berg, Semin. Arthritis Rheum., 30(5S-2):7-16, 2001.
Vannice and Levinson, J. Virology, 62:1305, 1988.
Varmus et al., Cell, 25:23-36, 1981.
Vasseur et al., Proc Natl. Acad. Sci. USA, 77:1068, 1980.
Wagner et al., Proc. Natl. Acad. Sci. USA 87(9):3410-3414, 1990.
Wang and Calame, Cell, 47:241, 1986.
Weber et al., Cell, 36:983, 1984.
Weinberger et al. Mol. Cell. Biol., 8:988, 1984.
Winoto and Baltimore, Cell, 59:649, 1989.
Wong et al., Gene, 10:87-94, 1980.
Wowk and Trapani, Microbes. Infect., 6:752-758, 2004.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang et al., Proc Natl. Acad. Sci. USA, 87:9568-9572, 1990.
Yutzey et al. Mol. Cell. Biol., 9:1397, 1989.
Zelenin et al., FEBS Lett., 280:94-96, 1991.
Zeng et al., J. Exp. Med., 201:139-148, 2005.

1. A method of generating a cytotoxic Granzyme B-producing B cell comprising contacting a B cell with IL-21 and one or more second agent selected from the group consisting of a TLR agonist, a cytokine, an antigen, anti-idiotype antibody, or an agent that cross-links surface immunoglobulin.

2. The method of claim 1, wherein the B cell is a malignant B cell.

3. The method of claim 57, wherein the TLR agonist is CpG ODN, immunostimulatory DNA, immunostimulatory RNA, immunostimulatory oligonucleotides, Imiquimod, Resiquimod, Loxribine, Flagellin, FSL-1 or LPS.

4.-6. (canceled)

7. The method of claim 1, wherein contacting comprises administration of IL-21 and said second agent to a subject.

8. The method of claim 7, wherein administration is systemic or intranodal.

9. The method of claim 7, wherein said subject suffers from cancer.

10. The method of claim 9, wherein said cancer is a B cell malignancy.

11. The method of claim 1, wherein contacting occurs in vitro.

12. The method of claim 11, further comprising administering said cytotoxic B cells to a subject.

13. The method of claim 12, wherein said subject suffers from cancer.

14. The method of claim 13, wherein said cancer is a B cell malignancy.

15. The method of claim 7, wherein said subject suffers from an infectious disease.

16. (canceled)

17. The method of claim 11, wherein said subject suffers from an infectious disease.

18. (canceled)

19. The method of claim 7, wherein said subject suffers from an autoimmune or hyperimmune disease.

20. The method of claim 19, wherein said disease is systemic lupus erythematosus; rheumatoid arthritis; Sjögren's syndrome; systemic sclerosis; polymyositis; grave's disease; myasthenia gravis; autoimmune diabetes (juvenile diabetes, diabetes type I); mononucleosis; Hyper-IgM, -IgD, -IgE syndrome; an anaphylactic reaction; a disease of excess or aberrant cytokine production; an auto-destructive immune response following infection with virus, bacteria, fungi or parasites; or an auto-destructive immune response following antibiotic, antiviral, anti-fungal or anti parasitic therapy.

21. (canceled)

22. The method of claim 20, further comprising treatment of said subject with a standard autoimmune disease therapy or a standard hyperimmune disease therapy.

23. (canceled)

24. The method of claim 11, wherein said subject suffers from an autoimmune disease or hyperimmune disease.

25.-28. (canceled)

29. A method of a generating an immune response in a subject comprising providing to said subject a cytotoxic Granzyme B-producing B cell.

30.-32. (canceled)

33. A method of inhibiting a T-regulatory response comprising providing to said subject a cytotoxic Granzyme B-producing B cell.

34.-56. (canceled)

57. The method of claim 1, wherein the second agent is a TLR agonist.
